(19) United States
(12) Patent Application Publication
(10) Pub. No.: US 2024/0167066 A1
(43) Pub. Date: May 23, 2024

(54) METHOD FOR PRODUCING DEMETHYLATED COMPOUND

(71) Applicant: DAICEL CORPORATION, Osaka-shi, Osaka (JP)

(72) Inventors: Akira OTANI, Tokyo (JP); Hiroaki YAMAMOTO, Tokyo (JP); Kazuya MITSUHASHI, Tokyo (JP); Takafumi MITACHI, Tokyo (JP); Satoru FUKIYA, Sapporo-shi (JP); Atsushi YOKOTA, Sapporo-shi (JP)

(73) Assignee: DAICEL CORPORATION, Osaka-shi, Osaka (JP)

(21) Appl. No.: 18/283,610

(22) PCT Filed: Mar. 24, 2022
(86) PCT No.: PCT/JP2022/013830

§ 371 Date: Sep. 22, 2023

(30) Foreign Application Priority Data
Mar. 24, 2021 (JP) 2021-050831

(51) Int. Cl. C12P 17/06; C12N 1/20; C12N 9/06; C12N 9/10; C12P 7/22; C12P 17/12; C12R 1/185; C12R 1/22; C12R 1/225; C12R 1/46

(52) U.S. Cl. CPC C12P 17/06; C12N 1/205; C12N 9/003; C12N 9/1014; C12P 7/22; C12P 17/12; C12R 2001/185; C12R 2001/22; C12R 2001/225; C12R 2001/46; C12Y 201/02001

(57) ABSTRACT

An object of the present disclosure is at least to provide a technique for promoting elimination of a methyl group(s) of a methoxy group(s) in causing a microorganism having a demethylation ability of eliminating a methyl group(s) of a methoxy group(s) from a compound with the methoxy group(s) in a side chain(s) to produce a demethylated compound in which a methyl group(s) of a methoxy group(s) has eliminated from a compound with the methoxy group(s) in a side chain(s). The issue is solved by a method for producing a demethylated compound, comprising co-culturing, in a solution containing a compound with a methoxy group(s) in a side chain(s), a microorganism having a demethylation ability of eliminating a methyl group(s) of a methoxy group(s) from a compound with the methoxy group(s) in a side chain(s), and a microorganism having an activity to promote the demethylation, to produce the demethylated compound in which a methyl group(s) of a methoxy group(s) has eliminated from the compound with the methoxy group(s) in the side chain(s).

### BACKGROUND OF THE INVENTION

### Field of the Invention

The present disclosure relates to a production method for producing a demethylated compound. More specifically, the present disclosure relates to a method for producing a demethylated compound in which a methyl group(s) of a methoxy group(s) has eliminated from a compound with the methoxy group(s) in a side chain(s).

### Description of the Related Art

As the demethylated compound in which a methyl group(s) of a methoxy group(s) has eliminated from a compound with the methoxy group(s) in a side chain(s), there are a large number of compounds that exhibit useful actions in a living body and compounds that can be used as raw materials thereof.

For example, a polyphenol with a methoxy group(s) in a side chain(s) is contained in a plant. Specific examples of the polyphenol with a methoxy group(s) in a side chain(s) include isoxanthohumol, glycitein, hesperetin, scoparone, and paeonol.

Examples of the demethylated polyphenol in which a methyl group(s) of the methoxy group(s) therefrom has eliminated include 8-prenylnaringenin, 6-hydroxydaidzein, eriodictyol, esculetin, and 4-acetylresorcinol.

For example, 8-prenylnaringenin produced by demethylating isoxanthohumol (a kind of flavanone) is known to have an estrogen-like activity and a disuse muscle atrophy inhibitory activity (Patent Document 1).

It has been reported that the Eubacterium limosum ATCC 8486 strain and the Blautia producta ATCC 27340 strain (formerly Peptostreptococcus productus ATCC 27340 strain) demethylate isoxanthohumol to produce 8-prenylnaringenin (Patent Documents 2 and 3).

In addition, 6-hydroxydaidzein produced by demethylating glycitein (a kind of isoflavone) can be a raw material for equol having an estrogen-like activity (Patent Document 4). Equol has a strong female hormone-like physiological action, and thus it has been proposed to use equol for prevention and improvement of menopausal symptoms and osteoporosis (Patent Document 5), prevention and treatment of skin aging and wrinkles (Patent Document 6), alleviation of allergic symptoms (Patent Document 7), and the like.

It has been reported that the Blautia coccoides JCM 1395 strain, the Blautia schinkii DSM 10518 strain, and microorganisms belonging to the Eubacterium limosum demethylate glycitein to produce 6-hydroxydaidzein (Patent Documents 4 and 8).

It has been reported that eriodictyol, which is demethylated hesperetin (a kind of flavanone), enhances an anticancer effect of EGCG by promoting activation of Akt, which plays a role in a 67LR-dependent cell-killing induction pathway, and exhibits a synergistic effect in an inhibitory effect on body fat accumulation and a preventive effect on abnormal lipid metabolism of green teas (Non-Patent Document 1).

It has been reported that the Blautia sp. MRG-PMF1 strain demethylates hesperetin to produce eriodictyol (Non-Patent Document 2).

Esculetin produced from scoparone (a kind of coumarin) with two methoxy groups in side chains by eliminating methyl groups of the two methoxy groups is blended in cosmetics, anti-inflammatory external skin preparations, anti-obesity agents, or the like (Patent Documents 9 to 11).

4-acetylresorcinol obtained by demethylating paeonol (a kind of simple phenols) is a compound useful as a synthetic intermediate of 2,4-dihydroxy-3-propylacetophenone, which is a raw material of a therapeutic agent for allergic diseases, or as a raw material of a photosensitive material or a sunscreen cosmetic (Patent Document 12).

### PRIOR ART DOCUMENTS

### Patent Documents

[Patent Document 1]: JP2013-35811A
[Patent Document 2]: JP2008-532558T
[Patent Document 3]: JP2020-115858A
[Patent Document 4]: JP 2020-058319A
[Patent Document 5]: JP2001-523258T
[Patent Document 6]: JP2002-511860T
[Patent Document 7]: JP4479505B
[Patent Document 8]: JP2010-104241A
[Patent Document 9]: JP2007-161727A
[Patent Document 10]: JP2006-28094A
[Patent Document 11]: JP2009-292811A
[Patent Document 12]: JPH08-225484A

### Non-Patent Documents

[Non-Patent Document 1]: Journal of Japan Society of Nutrition and Food Sciences, Vol. 72, No. 5, 205-210 (2019)
[Non-Patent Document 2]: S. Burapan, et al., Journal of Agric. Food Chem., 65, 1620-1629 (2017)

### SUMMARY OF THE INVENTION

### Problems to be Solved by the Invention

An object of the present disclosure is at least to provide a technique for promoting elimination of a methyl group(s) of a methoxy group(s) in causing a microorganism having a demethylation ability of eliminating a methyl group(s) of a methoxy group(s) from a compound with the methoxy group(s) in a side chain(s) to produce a demethylated compound in which a methyl group(s) of a methoxy group(s) has eliminated from a compound with the methoxy group(s) in a side chain(s).

### Means for Solving the Problems

<1> A method for producing a demethylated compound, comprising co-culturing, in a solution containing a compound with a methoxy group(s) in a side chain(s), a microorganism having a demethylation ability of eliminating a methyl group(s) of a methoxy group(s) from a compound with the methoxy group(s) in a side chain(s), and a microorganism having an activity to promote the demethylation, to produce the demethylated compound in which a methyl group(s) of a methoxy group(s) has eliminated from the compound with the methoxy group(s) in the side chain(s).
<2> The method according to <1>, wherein the microorganism having the activity to promote the demethylation is one or more microorganisms selected from the group consisting of a microorganism belonging to lactic acid bacterium, a microorganism belonging to the genus Akkermansia, a microorganism belonging to the genus Anaerofustis, a microorganism belonging to the genus Anaerotruncus, a microorganism belonging to the genus Arcobacter, a microorganism belonging to the genus Bacteroides, a microorganism belonging to the genus Clostridium, a microorganism belonging to the genus Coprobacillus, a microorganism belonging to the genus Dielina, a microorganism belonging to the genus Escherichia, a microorganism belonging to the genus Eubacterium, a microorganism belonging to the genus Faecalicoccus, a microorganism belonging to the genus Finegoldia, a microorganism belonging to the genus Hungatella, a microorganism belonging to the genus Intestinimonas, a microorganism belonging to the genus Parascardovia, a microorganism belonging to the genus Prevotella, a microorganism belonging to the genus Solobacterium, a microorganism belonging to the genus Sutterella, a microorganism belonging to the genus Bifidobacterium, a microorganism belonging to the genus Anaerostipes, a microorganism belonging to the genus Chitinophaga, a microorganism belonging to the genus Citrobacter, a microorganism belonging to the genus Clostridioides, a microorganism belonging to the genus Cryptobacterium, a microorganism belonging to the genus Edwardsiella, a microorganism belonging to the genus Klebsiella, a microorganism belonging to the genus Lacrimispora, a microorganism belonging to the genus Megasphaera, a microorganism belonging to the genus Parabacteroides, a microorganism belonging to the genus Providencia, a microorganism belonging to the genus Ruminococcus, and a microorganism belonging to the genus Yersinia.
<3> The method according to <2>, wherein the microorganism belonging to lactic acid bacterium is one or more microorganisms selected from the group consisting of a microorganism belonging to the genus Carnobacterium, a microorganism belonging to the genus Enterococcus, a microorganism belonging to the genus Fructobacillus, a microorganism belonging to the genus Lactobacillus, a microorganism belonging to the genus Lactococcus, a microorganism belonging to the genus Leuconostoc, a microorganism belonging to the genus Oenococcus, a microorganism belonging to the genus Pediococcus, a microorganism belonging to the genus Sporolactobacillus, a microorganism belonging to the genus Streptococcus, a microorganism belonging to the genus Tetragenococcus, and a microorganism belonging to the genus Weissella.
<4> The method according to any one of <1> to <3>, wherein the compound with the methoxy group(s) in the side chain(s) is a polyphenol with a methoxy group(s) in a side chain(s), and the demethylated compound in which the methyl group(s) of the methoxy group(s) has eliminated is a demethylated polyphenol in which a methyl group(s) of the methoxy group(s) has eliminated.
<5> The method according to <4>, wherein a combination of the polyphenol with the methoxy group(s) in the side chain(s) and the demethylated polyphenol in which the methyl group(s) of the methoxy group(s) has eliminated is one or more combinations selected from the group consisting of
a combination of flavanone with a methoxy group(s) in a side chain(s) and demethylated flavanone in which a methyl group(s) of the methoxy group(s) has eliminated,
a combination of isoflavone with a methoxy group(s) in a side chain(s) and demethylated isoflavone in which a methyl group(s) of the methoxy group(s) has eliminated,
a combination of coumarin with a methoxy group(s) in a side chain(s) and demethylated coumarin in which a methyl group(s) of the methoxy group(s) has eliminated, and
a combination of simple phenols with a methoxy group(s) in a side chain(s) and demethylated simple phenols in which a methyl group(s) of the methoxy group(s) has eliminated.
<6> The method according to <5>, wherein the combination of the flavanone with the methoxy group(s) in the side chain(s) and the demethylated flavanone in which the methyl group(s) of the methoxy group(s) has eliminated is one or more combinations selected from the group consisting of a combination of isoxanthohumol and 8-prenylnaringenin, and a combination of hesperetin and eriodictyol.
<7> The method according to <5> or <6>, wherein the isoflavone with the methoxy group(s) in the side chain(s) is glycitein, and the demethylated isoflavone in which the methyl group(s) of the methoxy group(s) has eliminated is 6-hydroxydaidzein.
<8> The method according to any one of <5> to <7>, wherein the coumarin with the methoxy group(s) in the side chain(s) is scoparone, and the demethylated coumarin in which the methyl group(s) of the methoxy group(s) has eliminated is one or more compounds selected from the group consisting of esculetin, scopoletin, and isoscopoletin.
<9> The method according to any one of <5> to <8>, wherein the simple phenols with the methoxy group(s) in the side chain(s) is paeonol, and the demethylated simple phenols in which the methyl group(s) of the methoxy group(s) has eliminated is 4-acetylresorcinol.
<10> The method according to any one of <1> to <9>, wherein the microorganism having the activity to promote the demethylation is a microorganism having an activity to promote regeneration of tetrahydrofolic acid from 5-methyltetrahydrofolate.
<11> The method according to <10>, wherein the microorganism having the activity to promote regeneration of tetrahydrofolic acid from 5-methyltetrahydrofolate is a microorganism producing dihydrofolate reductase-thymidylate synthase (E.C.1.5.1.3) and/or glycine hydroxymethyltransferase (E.C.2.1.2.1).
<12> A composition for promoting production of a demethylated compound in which a methyl group(s) of a methoxy group(s) has eliminated from a compound with the methoxy group(s) in a side chain(s), comprising a microorganism having a demethylation ability of eliminating a methyl group(s) of a methoxy group(s) from the compound with the methoxy group(s) in the side chain(s), and a microorganism having an activity to promote the demethylation.

### Effect of the Invention

The present disclosure can exhibit at least an effect of providing a technique for promoting elimination of a methyl group(s) of a methoxy group(s) in causing a microorganism having a demethylation ability of eliminating a methyl group(s) of a methoxy group(s) from a compound with the methoxy group(s) in a side chain(s) to produce a demethylated compound in which a methyl group(s) of a methoxy group(s) from a compound with the methoxy group(s) in a side chain(s), and can exhibit an effect of providing a useful microorganism therefor. As a result, in a method for producing a demethylated compound comprising causing a microorganism having a demethylation ability of eliminating a methyl group(s) of a methoxy group(s) from a compound with the methoxy group(s) in a side chain(s) to produce a demethylated compound in which a methyl group(s) of a methoxy group(s) is eliminated from the compound with the methoxy group(s) in a side chain(s). The present disclosure can exhibit an effect of promoting the elimination of the methyl group(s) of the methoxy group(s) to efficiently produce the demethylated compound.

### DESCRIPTION OF THE EMBODIMENTS

Note that each of the configurations, combinations thereof, and the like in each of the embodiments are an example, and various additions, omissions, substitutions, and other changes may be made as appropriate without departing from the spirit of the present disclosure. The present disclosure is not limited by the embodiments and is limited only by the claims.

Esculetin may be referred to as 6,7-dihydroxycoumarin or the like.

4-Acetylresorcinol may be referred to as 2,4-dihydroxyacetophenone, 2′,4′-dihydroxyacetophenone, resacetophenone, 2,4-DHAP, or the like.

Eriodictyol may be referred to as (S)-3′,4′,5,7-tetrahydroxyflavanone or the like.

In the present disclosure, a microorganism assigned a JCM number is stored in the Japan Collection of Microorganisms (National Research and Development Agency, Institute of Physical and Chemical Research, Bioresource Center, Microbe Division, zip code: 305-0074, address: 3-1-1 Koyadai, Tsukuba-shi, Ibaraki) and available from the organization.

A microorganism assigned a DSM number is stored in the DSMZ (Deutsche Sammlung von Mikroorganismen und Zellkulturen GmbH, address: Inhoffenstraβe 7B, 38124 Braunschweig. Germany) and available from the organization.

A microorganism assigned an ATCC number is stored in the American Type Culture Collection (address: 12301 Parklawn Drive, Rockville, Maryland. 20852, United States of America) and available from the organization.

A microorganism assigned an NBRC number is stored in the NITE Biological Resource Center (NBRC) of the National Institute of Technology and Evaluation (NITE) (zip code: 292-0818, address: 2-5-8 Kazusakamatari, Kisarazu-shi, Chiba) and available from the organization.

A microorganism assigned an NRIC number is stored in the Tokyo University of Agriculture Microorganisms Resource Center (zip code: 156-8502, address: 1-1-1 Sakuragaoka, Setagaya-ku, Tokyo), and available from the organization.

A microorganism assigned an IFO number is stored in the NITE Biological Resource Center (NBRC) of the National Institute of Technology and Evaluation (NITE) (zip code: 292-0818, address: 2-5-8 Kazusakamatari, Kisarazu-shi, Chiba) and available from the organization.

A microorganism assigned an AHU number is stored in the Laboratory of Applied Bacteriology of the Research Faculty of Agriculture, Hokkaido University (zip code: 060-8589, address: 9-chome, Kita9jonishi, Kita-ku, Sapporo-shi, Hokkaido) and available from the organization.

A microorganism assigned an IAM number is stored in the Japan Collection of Microorganisms (National Research and Development Agency. Institute of Physical and Chemical Research, Bioresource Center, Microbe Division, zip code: 305-0074, address: 3-1-1 Koyadai, Tsukuba-shi, Ibaraki) and available from the organization.

A microorganism assigned an NCIMB number is stored in the NCIMB Research Institute (The National Collections of Industrial, Food and Marine Bacteria, Ltd., address: Ferguson Building, Craibstone Estate, Bucksburn, Aberdeen, AB21 9YA, Scotland, UK) and available from the organization.

Method for Producing Demethylated Compound

An aspect of the present disclosure is method for producing a demethylated compound, comprising co-culturing, in a solution containing a compound with a methoxy group(s) in a side chain(s), a microorganism having a demethylation ability of eliminating a methyl group(s) of a methoxy group(s) from a compound with the methoxy group(s) in a side chain(s), and a microorganism having an activity to promote the demethylation, to produce the demethylated compound in which a methyl group(s) of a methoxy group(s) has eliminated from the compound with the methoxy group(s) in the side chain(s).

Compound with Methoxy Group in Side Chain

A compound with a methoxy group(s) in a side chain(s) may have one methoxy group or a plurality of methoxy groups. In either case, a raw material thereof is not particularly limited.

Examples of the compound with a methoxy group(s) in a side chain(s) include a polyphenol with a methoxy group(s) in a side chain(s), a terpenoid with a methoxy group(s) in a side chain(s), and an alkaloid with a methoxy group(s) in a side chain(s).

Examples of the polyphenol with a methoxy group(s) in a side chain(s) include phenolic acid with a methoxy group(s) in a side chain(s), lignan with a methoxy group(s) in a side chain(s), chroman with a methoxy group(s) in a side chain(s), coumarin with a methoxy group(s) in a side chain(s), flavonoid with a methoxy group(s) in a side chain(s), xanthone with a methoxy group(s) in a side chain(s), and simple phenols with a methoxy group(s) in a side chain(s).

Examples of the phenolic acid with a methoxy group(s) in a side chain(s) include ferulic acid (having one methoxy group in a side chain), anisic acid (having one methoxy group in a side chain), vanillic acid (having one methoxy group in a side chain), and syringic acid (having two methoxy groups in side chains).

Examples of the lignan with a methoxy group(s) in a side chain(s) include pinoresinol (having two methoxy groups in side chains) and secoisolariciresinol (having two methoxy groups in side chains).

Examples of the chroman with a methoxy group(s) in a side chain(s) include 6-methoxychroman (having one methoxy group in a side chain), 2-methoxychroman (having one methoxy group in a side chain), and 5-methoxychroman (having one methoxy group in a side chain).

Examples of the coumarin with a methoxy group(s) in a side chain(s) include scoparone (having two methoxy groups in side chains), scopoletin (having one methoxy group in a side chain), and isoscopoletin (having one methoxy group in a side chain).

Examples of the flavonoid with a methoxy group(s) in a side chain(s) include anthocyanidin with a methoxy group(s) in a side chain(s), flavan with a methoxy group(s) in a side chain(s), flavanol with a methoxy group(s) in a side chain(s) (also referred to as “catechin with a methoxy group(s) in a side chain(s)”), flavone with a methoxy group(s) in a side chain(s), flavonol with a methoxy group(s) in a side chain(s), flavanone with a methoxy group(s) in a side chain(s), isoflavone with a methoxy group(s) in a side chain(s), and chalcone with a methoxy group(s) in a side chain(s).

Examples of the anthocyanidin with a methoxy group(s) in a side chain(s) include malvidin (having two methoxy groups in side chains) and peonidin (having one methoxy group in a side chain).

Examples of the flavan with a methoxy group(s) in a side chain(s) include 4′-methoxyflavan (having one methoxy group in a side chain), 3′-methoxyflavan (having one methoxy group in a side chain), and 7-methoxyflavan (having one methoxy group in a side chain).

Examples of the flavanol with a methoxy group(s) in a side chain(s) include 3′-O-methylcatechin (having one methoxy group in a side chain), 4′-O-methylepicatechin (having one methoxy group in a side chain), and 4′-O-methylepigallocatechin (having one methoxy group in a side chain).

Examples of the flavone with a methoxy group(s) in a side chain(s) include nobiletin (having six methoxy groups in side chains), sinensetin (having five methoxy groups in side chains), tangeretin (having five methoxy groups in side chains), and wogonin (having one methoxy group in a side chain).

Examples of the flavonol with a methoxy group(s) in a side chain(s) include patuletin (having one methoxy group in a side chain), tamarixetin (having one methoxy group in a side chain), syringetin (having two methoxy groups in side chains), and izalpinin (having one methoxy group in a side chain).

Examples of the flavanone with a methoxy group(s) in a side chain(s) include isoxanthohumol (having one methoxy group in a side chain) and hesperetin (having one methoxy group in a side chain).

Examples of the isoflavone with a methoxy group(s) in a side chain(s) include glycitein (having one methoxy group in a side chain), biochanin (having one methoxy group in a side chain), formononetin (having one methoxy group in a side chain), and tectorigenin (having one methoxy group in a side chain).

Examples of the chalcone with a methoxy group(s) in a side chain(s) include xanthohumol (having one methoxy group in a side chain).

Examples of the xanthone with a methoxy group(s) in a side chain(s) include α-mangostin (having one methoxy group in a side chain) and β-mangostin (having two methoxy groups in side chains).

Examples of the simple phenols with a methoxy group(s) in a side chain(s) include paeonol (having one methoxy group in a side chain) and anisole (having one methoxy group in a side chain).

Demethylated Compound

In the present disclosure, a compound in which a methyl group(s) of a methoxy group(s) is eliminated from a “compound with the methoxy group(s) in a side chain(s)” in the above step may be referred to as a “demethylated compound”.

Note that in the present disclosure, in a case where the demethylated compound is produced from a compound with one methoxy group in a side chain, the demethylated compound may be produced by elimination of a methyl group of the one methoxy group, and in a case where the demethylated compound is produced from a compound with a plurality of methoxy groups, the demethylated compound may be produced by elimination of a methyl group of one methoxy group among the plurality of methoxy groups, may be produced by elimination of methyl groups of a plurality of methoxy groups (neither one methoxy group nor all methoxy groups) among the plurality of methoxy groups, or may be produced by elimination of methyl groups of all the methoxy groups among the plurality of methoxy groups.

In addition, in a case where methyl groups of a plurality of methoxy groups (neither one methoxy group nor all methoxy groups) are eliminated from a compound with a plurality of methoxy groups, a methoxy group(s) remains in the produced demethylated compound, and thus the produced demethylated compound can be used as the “compound with a methoxy group(s) in a side chain(s)”. For example, in a case where a methyl group of one methoxy group is eliminated from a compound with three methoxy groups, two methoxy groups remain in the produced demethylated compound, and thus the produced demethylated compound can be used as the “compound having a methoxy group(s) in a side chain(s)”.

Furthermore, in regard to the above description that the compound produced by elimination of a methyl group(s) of a methoxy group(s) from a “compound with the methoxy group(s) in a side chain(s)” may be referred to as a “demethylated compound”, specific examples of the “compound with a methoxy group(s) in a side chain(s)” may also be referred to in the same manner.

For example, a product produced by elimination of a methyl group(s) of a methoxy group(s) from a “polyphenol with the methoxy group(s) in a side chain(s)” may be referred to as a “demethylated polyphenol”.

Similarly, a product produced by elimination of a methyl group(s) of a methoxy group(s) from a “phenolic acid with the methoxy group(s) in a side chain(s)” may be referred to as a “demethylated phenolic acid”.

Similarly, a product produced by elimination of a methyl group(s) of a methoxy group(s) from a “lignan with the methoxy group(s) in a side chain(s)” may be referred to as a “demethylated lignan”.

Similarly, a product produced by elimination of a methyl group(s) of a methoxy group(s) from a “chroman with the methoxy group(s) in a side chain(s)” may be referred to as a “demethylated chroman”.

Similarly, a product produced by elimination of a methyl group(s) of a methoxy group(s) from a “coumarin with the methoxy group(s) in a side chain(s)” may be referred to as a “demethylated coumarin”.

Similarly, a product produced by elimination of a methyl group(s) of a methoxy group(s) from a “flavonoid with the methoxy group(s) in a side chain(s)” may be referred to as a “demethylated flavonoid”.

Similarly, a product produced by elimination of a methyl group(s) of a methoxy group(s) from an “anthocyanidin with the methoxy group(s) in a side chain(s)” may be referred to as a “demethylated anthocyanidin”.

Similarly, a product produced by elimination of a methyl group(s) of a methoxy group(s) from a “flavan with the methoxy group(s) in a side chain(s)” may be referred to as a “demethylated flavan”.

Similarly, a product produced by elimination of a methyl group(s) of a methoxy group(s) from a “flavanol with the methoxy group(s) in a side chain(s)” may be referred to as a “demethylated flavanol”.

Similarly, a product produced by elimination of a methyl group(s) of a methoxy group(s) from a “flavone with a methoxy group(s) in a side chain(s)” may be referred to as a “demethylated flavone”.

Similarly, a product produced by elimination of a methyl group(s) of a methoxy group(s) from a “flavonol with the methoxy group(s) in a side chain(s)” may be referred to as a “demethylated flavonol”.

Similarly, a product produced by elimination of a methyl group(s) of a methoxy group(s) from a “flavanone with the methoxy group(s) in a side chain(s)” may be referred to as a “demethylated flavanone”.

Similarly, a product produced by elimination of a methyl group(s) of a methoxy group(s) from an “isoflavone with the methoxy group(s) in a side chain(s)” may be referred to as a “demethylated isoflavone”.

Similarly, a product produced by elimination of a methyl group(s) of a methoxy group(s) from a “chalcone with the methoxy group(s) in a side chain(s)” may be referred to as a “demethylated chalcone”.

Similarly, a product produced by elimination of a methyl group(s) of a methoxy group(s) from a “xanthone with the methoxy group(s) in a side chain(s)” may be referred to as a “demethylated xanthone”.

Similarly, a product produced by elimination of a methyl group(s) of a methoxy group(s) from “simple phenols with the methoxy group(s) in a side chain(s)” may be referred to as “demethylated simple phenols”.

Similarly, a product produced by elimination of a methyl group(s) of a methoxy group(s) from a “terpenoid with the methoxy group(s) in a side chain(s)” may be referred to as a “demethylated terpenoid”.

Similarly, a product produced by elimination of a methyl group(s) of a methoxy group(s) from an “alkaloid with the methoxy group(s) in a side chain(s)” may be referred to as a “demethylated alkaloid”.

Examples of the demethylated phenolic acid include:
caffeic acid produced by elimination of a methyl group of one methoxy group of ferulic acid (having one methoxy group in a side chain);
salicylic acid produced by elimination of a methyl group of one methoxy group of anisic acid (having one methoxy group in a side chain).
protocatechuic acid produced by elimination of a methyl group of one methoxy group of vanillic acid (having one methoxy group in a side chain); and
3-O-methylgallic acid produced by elimination of a methyl group of one methoxy group and gallic acid produced by elimination of methyl groups of the two methoxy groups, of syringic acid (having two methoxy groups in side chains).

Examples of the demethylated lignan include:
1,2-benzene diol produced by elimination of a methyl group of one methoxy group and 3,3′-bisdemethylpinoresinol produced by elimination of methyl groups of the two methoxy groups, of pinoresinol (having two methoxy groups in side chains); and
O-demethylsecoisolariciresinol produced by elimination of a methyl group of one methoxy group and dihydroxy enterodiol produced by elimination of methyl groups of the two methoxy groups, of secoisolariciresinol (having two methoxy groups in side chains).

Examples of the demethylated chroman include:
6-hydroxychroman produced by elimination of a methyl group of one methoxy group of 6-methoxychroman (having one methoxy group in a side chain);
2-hydroxychroman produced by elimination of a methyl group of one methoxy group of 2-methoxychroman (having one methoxy group in a side chain); and
5-hydroxychroman produced by elimination of a methyl group of one methoxy group of 5-methoxychroman (having one methoxy group in the side chain).

Examples of the demethylated coumarin include:
scopoletin and isoscopoletin produced by elimination of a methyl group of one methoxy group and esculetin produced by elimination of methyl groups of the two methoxy groups, of scoparone (having two methoxy groups in side chains);
esculetin produced by elimination of a methyl group of one methoxy group of scopoletin (having one methoxy group in a side chain); and
esculetin produced by elimination of a methyl group of one methoxy group of isoscopoletin (having one methoxy group in a side chain).

Examples of the demethylated anthocyanidin include:
petunidin produced by elimination of a methyl group of one methoxy group and delphinidin produced by elimination of methyl groups of the two methoxy groups, of malvidin (having two methoxy groups in side chains); and
delphinidin produced by elimination of a methyl group of one methoxy group of peonidin (having one methoxy group in a side chain).

Examples of the demethylated flavan include:
4′-hydroxyflavan produced by elimination of a methyl group of one methoxy group of 4′-methoxyflavan (having one methoxy group in a side chain);
3′-hydroxyflavan produced by elimination of a methyl group of one methoxy group of 3′-methoxyflavan (having one methoxy group on a side chain); and
7-hydroxyflavan produced by elimination of a methyl group of one methoxy group of 7-methoxyflavan (having one methoxy group in a side chain).

Examples of the demethylated flavanol include:
catechin produced by elimination of a methyl group of one methoxy group of 3′-O-methylcatechin (having one methoxy group in a side chain);
epicatechin produced by elimination of a methyl group of one methoxy group of 4′-O-methylepicatechin (having one methoxy group in a side chain); and
epigallocatechin produced by elimination of a methyl group of one methoxy group of 4′-O-methylepigallocatechin (having one methoxy group in a side chain).

Examples of the demethylated flavone include:
4′-demethylnobiletin produced by elimination of a methyl group of one methoxy group, 3′,4′-dimethylnobiletin produced by elimination of methyl groups of two methoxy groups, sideritoflavone produced by elimination of methyl groups of three methoxy groups, leucangenin produced by elimination of methyl groups of four methoxy groups, 2-(3,4-dihydroxyphenyl)-5,6,8-trihydroxy-7-methoxy-4H-1-benzopyran-4-one produced by elimination of methyl groups of five methoxy groups, and vitellogenin produced by elimination of methyl groups of six methoxy groups, of nobiletin (having six methoxy groups in side chains);
4′-desmethylsinensetin produced by elimination of a methyl group of one methoxy group, 3′,4′-dihydroxy-5,6,7-trimethoxyflavone produced by elimination of methyl groups of two methoxy groups, 3′,4′,7′-trihydroxy-5,6-dimethyloxyflavone produced by elimination of methyl groups of three methoxy groups, carajuflavone produced by elimination of methyl groups of four methoxy groups, and 6-hydroxyluteolin produced by elimination of methyl groups of five methoxy groups, of sinensetin (having five methoxy groups in side chains);
4′-hydroxy-5,6,7,8-tetramethoxyflavone produced by elimination of a methyl group of one methoxy group, xanthomicrol produced by elimination of methyl groups of two methoxy groups, isotylmusin produced by elimination of methyl groups of three methoxy groups, 5,7,8-trihydroxy-2-(4-hydroxyphenyl)-6-methoxy-4H-1-benzopyran-4-one produced by elimination of methyl groups of four methoxy groups, nortangeretin produced by elimination of methyl groups of five methoxy groups, of tangeretin (having five methoxy groups in side chains); and
norwogonin produced by elimination of a methyl group of one methoxy group of wogonin (having one methoxy group in a side chain).

Examples of the demethylated flavonol include:
chelsetagetin produced by elimination of a methyl group of one methoxy group of patchouletin (having one methoxy group in a side chain);
quercetin produced by elimination of a methyl group of one methoxy group of tamarixetin (having one methoxy group in a side chain);
laricitrine produced by elimination of a methyl group of one methoxy group, and myricetin produced by elimination of methyl groups from two methoxy groups, of syringetine (having two methoxy groups in side chains); and
galangin produced by elimination of a methyl group of one methoxy group of izalpinin (having one methoxy group in a side chain).

Examples of the demethylated flavanone include;
8-prenylnaringenin produced by elimination of a methyl group of one methoxy group of isoxanthohumol (having one methoxy group in a side chain; and
eriodictyol produced by elimination of a methyl group of one methoxy group of hesperetin (having one methoxy group in a side chain).

Examples of the demethylated isoflavone include:
6-hydroxydaidzein produced by elimination of a methyl group of one methoxy group of glycitein (having one methoxy group in a side chain);
genistein produced by elimination of a methyl group of one methoxy group of biochanin (having one methoxy group in a side chain);
daidzein produced by elimination of a methyl group of one methoxy group of formononetin (having one methoxy group in a side chain); and
6-hydroxygenistein produced by elimination of a methyl group of one methoxy group of tectorigenin (having one methoxy group in a side chain).

Examples of the demethylated chalcone include:
demethylxanthohumol produced by elimination of a methyl group of one methoxy group of xanthohumol (having one methoxy group in a side chain).

Examples of the demethylated xanthone include:
γ-mangostin produced by elimination of a methyl group of one methoxy group of α-mangostin (having one methoxy group in a side chain); and
α-mangostin produced by elimination of a methyl group of one methoxy group, and γ-mangostin produced by elimination of methyl groups of two methoxy groups, of β-mangostin (having two methoxy groups in side chains).

Examples of the demethylated simple phenols include:
4-acetylresorcinol produced by elimination of a methyl group of one methoxy group of paeonol (having one methoxy group in a side chain); and
phenol produced by elimination of a methyl group of one methoxy group of anisole (having one methoxy group in a side chain).

Microorganism Having Demethylation Ability of Eliminating Methyl Group of Methoxy group from Compound with Methoxy Group in Side Chain

The microorganism having a demethylation ability of eliminating a methyl group(s) of a methoxy group(s) from a compound with the methoxy group(s) in a side chain(s), which is used in the present aspect, is not particularly limited as long as it has a demethylation ability of eliminating a methyl group(s) of a methoxy group(s) from a compound with the methoxy group(s) in a side chain(s).

The microorganism can be obtained by a usual screening method. For example, when a compound with a methoxy group(s) in a side chain(s) is used as a raw material and the microorganism is cultured in accordance with a usual culture method, a microorganism capable of producing a demethylated compound by eliminating a methyl group(s) of a methoxy group(s) from a compound with a methoxy group(s) in a side chain(s) can be obtained as the microorganism.

The microorganism is preferably a bacterium having a demethylation ability of eliminating a methyl group(s) of a methoxy group(s) from a compound with the methoxy group(s) in a side chain(s). The bacterium is preferably an enteric bacterium or the like.

Examples of the enteric bacterium include a microorganism belonging to the genus Blautia, a microorganism belonging to the genus Eubacterium, and a microorganism belonging to the genus Acetobacterium.

Examples of the microorganism belonging to the genus Blautia include a microorganism belonging to Blautia producta (e.g., ATCC 27340 strain and the like), a microorganism belonging to Blautia coccoides (e.g., JCM 1395 strain and the like), a microorganism belonging to Blautia schinkii (e.g., DSM 10518 strain and the like), a microorganism belonging to Blautia hominis (e.g., JCM 32276 strain and the like), Blautia sp. DC 3652 (NITE BP-02924) strain, Blautia sp. DC 3653 (NITE BP-02629) strain, Blautia sp. DC 3654 (NITE BP-02925) strain, and Blautia sp. MRG-PMF1 strain.

Examples of the microorganism belonging to the genus Eubacterium include a microorganism belonging to Eubacterium limosum (e.g., JCM 6421 strain, ATCC 8486 strain, JCM 6501 strain, JCM 9978 strain, and the like).

Examples of the microorganism belonging to the genus Acetobacterium include a microorganism belonging to Acetobacterium bakii (e.g., DSM 8239 strain and the like), a microorganism belonging to Acetobacterium dehalogenans (e.g., DSM 11527 strain and the like), a microorganism belonging to Acetobacterium wieringae (e.g., DSM 1911 strain and the like), and a microorganism belonging to Acetobacterium woodii (e.g., DSM 1030 strain and the like).

The Blautia sp. DC 3652 (NITE BP-02924) strain was internationally deposited with Patent Microorganisms Depositary, National Institute of Technology and Evaluation (NITE) (zip code: 292-0818, address: #122, 2-5-8 Kazusa Kamatari, Kisarazu-shi, Chiba) under accession No. NITE BP-02924 as of Mar. 20, 2019, under the Budapest Treaty.

The Blautia sp. DC 3653 (NITE BP-02629) strain was deposited in Japan with Patent Microorganisms Depositary, National Institute of Technology and Evaluation (NITE) (zip code: 292-0818, address: #122, 2-5-8 Kazusa Kamatari, Kisarazu-shi, Chiba) under accession No. NITE P-02629 as of Feb. 7, 2018, demanded to transfer to international deposit under the Budapest Treaty on Dec. 27, 2018, and the accession No. NITE BP-02629 was assigned.

The Blautia sp. DC 3654 (NITE BP-02925) strain was internationally deposited with Patent Microorganisms Depositary, National Institute of Technology and Evaluation (NITE) (zip code: 292-0818, address: #122, 2-5-8 Kazusa Kamatari, Kisarazu-shi, Chiba) under accession No. NITE BP-02925 as of Mar. 20, 2019, under the Budapest Treaty.

When the Blautia producta ATCC 27340 strain is taken as an example, in the present aspect, the Blautia producta ATCC 27340 strain is not limited to the deposited strain, and may be a strain substantially equivalent to the deposited strain. The “strain substantially equivalent” refers to a strain belonging to the same genus or species as the deposited strain and having a demethylation ability of eliminating a methyl group(s) of a methoxy group(s) from a compound with the methoxy group(s) in a side chain(s). The strain substantially equivalent also is a strain in which the base sequence of the 16S rRNA gene is 98.5% or greater, preferably 98.7% or greater, more preferably 99% or greater, and even more preferably 100% homologous to the base sequence of the 16S rRNA gene of the deposited strain. Furthermore, the deposited strain may be a strain that is grown from the deposited strain or a strain substantially equivalent thereto, by mutation treatment, genetic recombination, selection of a natural mutant strain, or the like, as long as the strain has a demethylation ability of eliminating a methyl group(s) of a methoxy group(s) from a compound with the methoxy group(s) in a side chain(s).

This also applies to the other deposited strains described above.

In the present aspect, one kind or two or more kinds of the microorganisms may be used, and one strain or two or more strains may be used.

Resting Body of Microorganism Having Demethylation Ability of Eliminating Methyl Group of Methoxy Group from Compound with Methoxy Group in Side chain

In the present aspect, the microorganism having a demethylation ability of eliminating a methyl group(s) of a methoxy group(s) from a compound with the methoxy group(s) in a side chain(s) includes a resting body thereof.

The “resting body” refers to a microorganism body obtained by removing culture medium components from a cultured microorganism by manipulations such as centrifugation, washing the microorganism with a salt solution or a buffer solution, and suspending the microorganism in the same liquid as the washing solution, the microorganism body being in a non-proliferative state. In the present aspect, the resting body refers to at least a microorganism body having a metabolic system that can eliminate a methyl group(s) of a methoxy group(s) from a compound with the methoxy group(s) in a side chain(s). In a case where the microorganism is a bacterium having a demethylation ability of eliminating a methyl group(s) of a methoxy group(s) from a compound with the methoxy group(s) in a side chain(s), the resting body is a resting bacterial body.

Examples of the salt solution include physiological saline. Examples of the buffer solution include a phosphate buffer solution, a tris-hydrochloric acid buffer solution, a citrate-phosphate buffer solution, a citrate buffer solution, a MOPS buffer solution, an acetate buffer solution, and a glycine buffer solution. In any case, the pH and concentration can be appropriately adjusted in accordance with a known method.

Microorganism Having Activity to Promote Demethylation

The microorganism having an activity to promote demethylation in the present aspect is a microorganism having an activity to promote demethylation of a microorganism having a demethylation ability of eliminating a methyl group(s) of a methoxy group(s) from a compound with the methoxy group(s) in a side chain(s).

The microorganism is not particularly limited as long as it has an activity to promote demethylation of a microorganism having a demethylation ability of eliminating a methyl group(s) of a methoxy group(s) from a compound with the methoxy group(s) in a side chain(s). The microorganism may be the same as or different from the microorganism having a demethylation ability of eliminating a methyl group(s) of a methoxy group(s) from a compound with the methoxy group(s) in a side chain(s).

The microorganism is preferably a bacterium having an activity to promote demethylation of the microorganism having a demethylation ability of eliminating a methyl group(s) of a methoxy group(s) from a compound with the methoxy group(s) in a side chain(s). The bacterium is preferably an enteric bacterium or the like.

Examples of the enteric bacterium include a microorganism belonging to lactic acid bacteria, a microorganism belonging to the genus Akkermansia, a microorganism belonging to the genus Anaerofustis, a microorganism belonging to the genus Anaerotruncus, a microorganism belonging to the genus Arcobacter, a microorganism belonging to the genus Bacteroides, a microorganism belonging to the genus Clostridium, a microorganism belonging to the genus Coprobacillus, a microorganism belonging to the genus Dielma, a microorganism belonging to the genus Escherichia, a microorganism belonging to the genus Eubacterium, a microorganism belonging to the genus Faecalicoccus, a microorganism belonging to the genus Finegoldia, a microorganism belonging to the genus Hungatella, a microorganism belonging to the genus Intestinimonas, a microorganism belonging to the genus Parascardovia, a microorganism belonging to the genus Prevotella, a microorganism belonging to the genus Solobacterium, a microorganism belonging to the genus Sutterella, a microorganism belonging to the genus Bifidobacterium, a microorganism belonging to the genus Anaerostipes, a microorganism belonging to the genus Chitinophaga, a microorganism belonging to the genus Citrobacter, a microorganism belonging to the genus Clostridioides, a microorganism belonging to the genus Cryptobacterium, a microorganism belonging to the genus Edwardsiella, a microorganism belonging to the genus Klebsiella, a microorganism belonging to the genus Lacrimispora, a microorganism belonging to the genus Megasphaera, a microorganism belonging to the genus Parabacteroides, a microorganism belonging to the genus Providencia, a microorganism belonging to the genus Ruminococcus, and a microorganism belonging to the genus Yersinia.

Examples of the microorganism belonging to lactic acid bacteria include a microorganism belonging to the genus Carnobacterium, a microorganism belonging to the genus Enterococcus, a microorganism belonging to the genus Fructobacillus, a microorganism belonging to the genus Lactobacillus, a microorganism belonging to the genus Lactococcus, a microorganism belonging to the genus Leuconostoc, a microorganism belonging to the genus Oenococcus, a microorganism belonging to the genus Pediococcus, a microorganism belonging to the genus Sporolactobacillus, a microorganism belonging to the genus Streptococcus, a microorganism belonging to the genus Tetragenococcus, and a microorganism belonging to the genus Weissella.

Examples of the microorganism belonging to the genus Carnobacterium include a microorganism belonging to the genus Carnobacterium divergens (for example, NBRC 15683 strain and the like).

Examples of the microorganism belonging to the genus Enterococcus include a microorganism belonging to Enterococcus avium (e.g., NITE BP-03387 strain, NITE BP-03386 strain, and the like), a microorganism belonging to Enterococcus caccae (e.g., DSM 19114 strain and the like), a microorganism belonging to Enterococcus faecalis subsp. liquefaciens (e.g., NRIC 1746 strain and the like), and a microorganism belonging to Enterococcus hirae (e.g., JCM 8717, JCM 8719 strain, NRIC 102 strain, NRIC 108 strain, and the like).

Examples of the microorganism belonging to the genus Fructobacillus include a microorganism belonging to Fructobacillus fructosus (e.g., NBRC 3516 strain and the like).

Examples of the microorganism belonging to the genus Lactobacillus include a microorganism belonging to Lactobacillus acetotolerans (e.g., JCM 3825 strain and the like), a microorganism belonging to Lactobacillus acidifarinae (e.g., NBRC 107156 strain and the like), a microorganism belonging to Lactobacillus acidophilus (e.g., IFO 13951 strain and the like), a microorganism belonging to Lactobacillus agilis (e.g., JCM 1187 strain and the like), a microorganism belonging to Lactobacillus algidus (e.g., JCM 10491 strain and the like), a microorganism belonging to Lactobacillus alimentarius (e.g., NBRC 106464 strain and the like), a microorganism belonging to Lactobacillus amylolyticus (e.g., JCM 12529 strain and the like), a microorganism belonging to Lactobacillus amylophilus (e.g., IFO 15881 strain and the like), a microorganism belonging to Lactobacillus amylotrophicus (e.g., JCM 1124 strain and the like), a microorganism belonging to Lactobacillus antri (e.g., JCM 15950 strain and the like), a microorganism belonging to Lactobacillus apodemi (e.g., JCM 16172 strain and the like), a microorganism belonging to Lactobacillus aquaticus (e.g., JCM 16869 strain and the like), a microorganism belonging to Lactobacillus aviarius subsp. Aviarius (e.g., NBRC 102162 strain and the like), a microorganism belonging to Lactobacillus bifermentans (e.g., JCM 1094 strain and the like), a microorganism belonging to Lactobacillus brantae (e.g., DSM 23927 strain and the like), a microorganism belonging to Lactobacillus brevis (e.g., NRIC 1037 strain and the like), a microorganism belonging to Lactobacillus buchneri (e.g., NRIC 1040 strain, NRIC 1079 strain, NRIC 1082 strain, and the like), a microorganism belonging to Lactobacillus camelliae (e.g., JCM 13995 strain and the like), a microorganism belonging to Lactobacillus capillatus (e.g., JCM 15044 strain and the like), a microorganism belonging to Lactobacillus casei (e.g., AHU 1055 strain and the like), a microorganism belonging to Lactobacillus ceti (e.g., JCM 15609 strain and the like), a microorganism belonging to Lactobacillus coleohominis (e.g., JCM 11550 strain and the like), a microorganism belonging to Lactobacillus collinoides (e.g., NRIC 1049 strain and the like), a microorganism belonging to Lactobacillus composti (e.g., JCM 14202 strain and the like), a microorganism belonging to Lactobacillus curvatus (e.g., NBRC 15884 strain and the like), a microorganism belonging to Lactobacillus delbrueckii (e.g., AHU 1056 strain, NBRC 102622 strain, and the like), a microorganism belonging to Lactobacillus delbrueckii subsp. delbruechii (e.g., IAM 1149 strain. IAM 1928 strain, IFO 3534 strain, and the like), a microorganism belonging to Lactobacillus delbrueckii subsp. indicus (e.g., JCM 15610 strain and the like), a microorganism belonging to Lactobacillus delbrueckii subsp. lactis (e.g., IFO 3073 strain, JCK 1557 strain, NRIC 1061 strain, and the like), a microorganism belonging to Lactobacillus diolivorans (e.g., NBRC 107869 strain and the like), a microorganism belonging to Lactobacillus equi (e.g., JCM 10991 strain and the like), a microorganism belonging to Lactobacillus equicursoris (e.g., JCM 14600 strain and the like), a microorganism belonging to Lactobacillus equigenerosi (e.g., JCM 14505 strain and the like), a microorganism belonging to Lactobacillus fabifermentans (e.g., DSM 21115 strain and the like), a microorganism belonging to Lactobacillus farraginis (e.g., JCM 14108 strain and the like), and a microorganism belonging to Lactobacillus floricola (e.g., JCM 16512 strain and the like).

In addition, examples thereof include a microorganism belonging to Lactobacillus florum (e.g., JCM 16035 strain and the like), a microorganism belonging to Lactobacillus fructivorans (e.g., NBRC 13954 strain, NRIC 224 strain, and the like), a microorganism belonging to Lactobacillus frumenti (e.g., JCM 11122 strain and the like), a microorganism belonging to Lactobacillus fuchuensis (e.g., JCM 11249 strain and the like), a microorganism belonging to Lactobacillus gasseri (e.g., JCM 1131 strain and the like), a microorganism belonging to Lactobacillus gastricus (e.g., JCM 15952 strain and the like), a microorganism belonging to Lactobacillus ghanensis (e.g., JCM 15611 strain and the like), a microorganism belonging to Lactobacillus graminis (e.g., JCM 9503 strain and the like), a microorganism belonging to Lactobacillus hammesii (e.g., JCM 16170 strain and the like), a microorganism belonging to Lactobacillus hamsteri (e.g., JCM 6256 strain and the like), a microorganism belonging to Lactobacillus harbinensis (e.g., NBRC 100982 strain and the like), a microorganism belonging to Lactobacillus hayakitensis (e.g., JCM 14209 strain and the like), a microorganism belonging to Lactobacillus hilgardii (e.g., strain DSM 20051 strain, NBRC 15886 strain, NRIC 1060 strain, and the like), a microorganism belonging to Lactobacillus hokkaidonensis (e.g., JCM 18461 strain and the like), a microorganism belonging to Lactobacillus hominis (e.g., DSM 23910 strain and the like), a microorganism belonging to Lactobacillus hordei (e.g., JCM 16179 strain and the like), a microorganism belonging to Lactobacillus iners (e.g., JCM 12513 strain and the like), a microorganism belonging to Lactobacillus ingluviei (e.g., JCM 12531 strain and the like), a microorganism belonging to Lactobacillus intestinalis (e.g., JCM 7548 strain and the like), a microorganism belonging to Lactobacillus iwatensis (e.g., JCM 18838 strain and the like), a microorganism belonging to Lactobacillus jensenii (e.g., JCM 15953 strain and the like), a microorganism belonging to Lactobacillus johnsonii (e.g., JCM 2012 strain and the like), a microorganism belonging to Lactobacillus kalixensis (e.g., JCM 15954 strain and the like), a microorganism belonging to Lactobacillus kefiranofaciens subsp. kefirgranum (e.g., JCM 8572 strain and the like), a microorganism belonging to Lactobacillus kefiri (e.g., NRIC 1693 strain and the like), a microorganism belonging to Lactobacillus kimchiensis (e.g., JCM 17702 strain and the like), a microorganism belonging to Lactobacillus kisonensis (e.g., JCM 15041 strain and the like), a microorganism belonging to Lactobacillus kitasatonis (e.g., JCM 1039 strain and the like), a microorganism belonging to Lactobacillus koreensis (e.g., JCM 16448 strain and the like), a microorganism belonging to Lactobacillus lactis (e.g., AHU 1059 strain and the like), a microorganism belonging to Lactobacillus leichmannii (e.g., AHU 1681 strain and the like), a microorganism belonging to Lactobacillus malefermentans (e.g., DSM 5705 strain, NRIC 1081 strain, and the like), a microorganism belonging to Lactobacillus mali (e.g., NRIC 1076 strain and the like), a microorganism belonging to Lactobacillus manihotivorans (e.g., JCM 12514 strain and the like), a microorganism belonging to Lactobacillus mindensis (e.g., NBRC 107162 strain and the like), a microorganism belonging to Lactobacillus mixtipabuli (e.g., JCM 19805 strain and the like), a microorganism belonging to Lactobacillus murinus (e.g., IFO 14221 strain and the like), and a microorganism belonging to Lactobacillus nagelii (e.g., JCM 12492 strain and the like).

Other examples thereof include a microorganism belonging to Lactobacillus namurensis (e.g., NBRC 107158 strain and the like), a microorganism belonging to Lactobacillus nantensis (e.g., NBRC 107153 strain and the like), a microorganism belonging to Lactobacillus nasuensis (e.g., JCM 17158 strain and the like), a microorganism belonging to Lactobacillus nenjiangensis (e.g., JCM 30919 strain and the like), a microorganism belonging to Lactobacillus oeni (e.g., JCM 18036 strain and the like), a microorganism belonging to Lactobacillus oligofermentans (e.g., JCM 16175 strain and the like), a microorganism belonging to Lactobacillus oris (e.g., JCM 11028 strain and the like), a microorganism belonging to Lactobacillus orvzae (e.g., JCM 18671 strain and the like), a microorganism belonging to Lactobacillus otakiensis (e.g., JCM 15040 strain and the like), a microorganism belonging to Lactobacillus ozensis (e.g., JCM 17196 strain and the like), a microorganism belonging to Lactobacillus panis (e.g., 3CM 11053 strain and the like), a microorganism belonging to Lactobacillus pantheris (e.g., NBRC 106106 strain and the like), a microorganism belonging to Lactobacillus parabrevis (e.g., NBRC 107154 strain and the like), a microorganism belonging to Lactobacillus parabuchneri (e.g., NBRC 107865 strain and the like), a microorganism belonging to Lactobacillus paracollinoides (e.g., JCM 11969 strain and the like), a microorganism belonging to Lactobacillus parafarraginis (e.g., JCM 14109 strain and the like), a microorganism belonging to Lactobacillus parakefiri (e.g., NBRC 15890 strain and the like), a microorganism belonging to Lactobacillus paralimentarius (e.g., NBRC 106466 strain, NBRC 107149 strain, NBRC 107152 strain, and the like), a microorganism belonging to Lactobacillus paraplantarum (e.g., NBRC 107151 strain and the like), a microorganism belonging to Lactobacillus paucivorans (e.g., JCM 18045 strain and the like), a microorganism belonging to Lactobacillus pentosiphilus (e.g., JCM 31145 strain and the like), a microorganism belonging to Lactobacillus penlosus (e.g., IFO 12011 strain, NBRC 106467 strain, and the like), a microorganism belonging to Lactobacillus perolens (e.g., JCM 12534 strain and the like), a microorganism belonging to Lactobacillus plantarum (e.g., DSM 13273 strain, IFO 3070 strain, NCIMB 8826 strain, NRIC 1068 strain, and the like), a microorganism belonging to Lactobacillus plantarum subsp. argentoratensis (e.g., NBRC 106468 strain and the like), a microorganism belonging to Lactobacillus plantarum subsp. plantarum (e.g., NBRC 15891 strain and the like), a microorganism belonging to Lactobacillus pobuzihii (e.g., NBRC 103219 strain, JCM 18084 strain, and the like), a microorganism belonging to Lactobacillus pontis (e.g., JCM 11051 strain and the like), a microorganism belonging to Lactobacillus porci (e.g., DSM 105804 strain and the like), a microorganism belonging to Lactobacillus porcinae (e.g., JCM 19617 strain and the like), a microorganism belonging to Lactobacillus rapi (e.g., NBRC 109618 strain and the like), a microorganism belonging to Lactobacillus rhamnosus (e.g., DSM 20021 strain, IFO 3425 strain, and the like), a microorganism belonging to Lactobacillus rossiae (e.g., JCM 16176 strain and the like), a microorganism belonging to Lactobacillus ruminis (e.g., NBRC 102161 strain and the like), a microorganism belonging to Lactobacillus saerimneri (e.g., NBRC 107826 strain and the like), and a microorganism belonging to Lactobacillus sakei subsp. carnosus (e.g., NBRC 107868 strain and the like).

Other examples thereof include a microorganism belonging to Lactobacillus salivarius subsp. salicinius (e.g., NRIC 1072 strain and the like), a microorganism belonging to Lactobacillus sanfranciscensis (e.g., JCM 5668 strain and the like), a microorganism belonging to Lactobacillus saniviri (e.g., JCM 17471 strain and the like), a microorganism belonging to Lactobacillus satsumensis (e.g., JCM 12392 strain and the like), a microorganism belonging to Lactobacillus secaliphilus (e.g., JCM 15613 strain and the like), a microorganism belonging to Lactobacillus senmaizukei (e.g., NBRC 103853 strain and the like), a microorganism belonging to Lactobacillus sharpeae (e.g., JCM 1186 strain and the like), a microorganism belonging to Lactobacillus siliginis (e.g., NBRC 101315 strain and the like), a microorganism belonging to Lactobacillus songhuajiangensis (e.g., JCM 30918 strain and the like), Lactobacillus sp. NRIC 1029 strain, a microorganism belonging to Lactobacillus spicheri (e.g., NBRC 107155 strain and the like), a microorganism belonging to Lactobacillus sucicola (e.g., JCM 15457 strain and the like), a microorganism belonging to Lactobacillus suebicus (e.g., JCM 9504 strain and the like), a microorganism belonging to Lactobacillus sunkit (e.g., JCM 15039 strain and the like), a microorganism belonging to Lactobacillus thailandensis (e.g., JCM 13996 strain and the like), a microorganism belonging to Lactobacillus tucceti (e.g., JCM 18037 strain and the like), a microorganism belonging to Lactobacillus ultunensis (e.g., JCM 16177 strain), a microorganism belonging to Lactobacillus uvarum (e.g., JCM 16870 strain and the like), a microorganism belonging to Lactobacillus vaccinostercus (e.g., NRIC 1075 strain and the like), a microorganism belonging to Lactobacillus vermoldensis (e.g., NBRC 106069 strain and the like), a microorganism belonging to Lactobacillus vini (e.g., JCM 14280 strain and the like), a microorganism belonging to Lactobacillus wasatchensis (e.g., DSM 29958 strain and the like), a microorganism belonging to Lactobacillus xiangfangensis (e.g., NBRC 108914 strain and the like), a microorganism belonging to Lactobacillus zeae (e.g., DSM 20178 strain and the like), and a microorganism belonging to Lactobacillus zymae (e.g., NBRC 107157 strain and the like).

Other examples thereof include a microorganism belonging to Lactobacillus chiayiensis (e.g., NBRC 112906 strain and the like), a microorganism belonging to Lactobacillus apinorum (e.g., DSM 26257 strain and the like), a microorganism belonging to Lactobacillus ixorae (e.g., NBRC 111239 strain and the like), a microorganism belonging to Lactobacillus kullabergensis (e.g., DSM 26262 strain and the like), a microorganism belonging to Lactobacillus mellifer (e.g., DSM 26254 strain and the like), a microorganism belonging to Lactobacillus modestisalitolerans (e.g., NBRC 107235 strain and the like), a microorganism belonging to Lactobacillus plajomi (e.g., NBRC 107333 strain and the like), and a microorganism belonging to Lactobacillus suantsalihabitans (e.g., NBRC 113532 strain and the like).

Examples of the microorganism belonging to the genus Lactococcus include a microorganism belonging to Lactococcus fujiensis (e.g., JCM 16395 strain and the like), a microorganism belonging to Lactococcus garvieae (e.g., NBRC 100934 strain and the like), a microorganism belonging to Lactococcus lactis subsp. lactis (e.g., NRIC 1074 strain, NRIC 1149 strain, and the like), a microorganism belonging to Lactococcus lactis subsp. tructae (e.g., DSM 21502 strain and the like), and a microorganism belonging to Lactococcus taiwanensis (e.g., NBRC 109049 strain and the like).

Examples of the microorganism belonging to the genus Leuconostoc include a microorganism belonging to Leuconostoc citreum (e.g., JCM 9698 strain and the like), a microorganism belonging to Leuconostoc dextranicum (e.g., AHU 1078 strain, IFO 3347 strain, and the like), a microorganism belonging to Leuconostoc lactis (e.g., IFO 12455 strain and the like), and a microorganism belonging to Leuconostoc mesenteroides subsp. cremoris (e.g., TAM 1087 strain, NRIC 1538 strain, and the like).

Examples of the microorganism belonging to the genus Oenococcus include a microorganism belonging to Oenococcus oeni (e.g., ATCC 27311 strain, DSM 20252 strain, and the like).

Examples of the microorganism belonging to the genus Pediococcus include a microorganism belonging to Pediococcus acidilactici (e.g., NRIC 1102 strain and the like), a microorganism belonging to Pediococcus argentinicus (e.g., JCM 30771 strain and the like), a microorganism belonging to Pediococcus cellicola (e.g., JCM 14152 strain and the like), a microorganism belonging to Pediococcus claussenii (e.g., JCM 18046 strain and the like), a microorganism belonging to Pediococcus damnosus (e.g., JCM 5886 strain and the like), a microorganism belonging to Pediococcus inopinatus (e.g., JCM 12518 strain and the like), a microorganism belonging to Pediococcus parvulus (e.g., JCM 5889 strain and the like), and a microorganism belonging to Pediococcus pentosaceus (e.g., IFO 3891 strain, NRIC 1106 strain, and the like).

Examples of the microorganism belonging to the genus Sporolactobacillus include a microorganism belonging to Sporolactobacillus inulinus (e.g., NRIC 1133 strain and the like).

Examples of the microorganism belonging to the genus Streptococcus include a microorganism belonging to Streptococcus alactolyticus (e.g., DSM 100950 strain and the like), a microorganism belonging to Streptococcus equinus (e.g., NRIC 1139 strain and the like), and a microorganism belonging to Streptococcus uberis (e.g., NRIC 1153 strain and the like).

Examples of the microorganism belonging to the genus Tetragenococcus include a microorganism belonging to Tetragenococcus halophilus subsp. halophilus (e.g., the NBRC 100498 strain and the like).

Examples of the microorganism belonging to the genus Weissella include a microorganism belonging to Weissella confusa (e.g., DSM 20196 strain, NBRC 106469 strain, and the like), and a microorganism belonging to Weissella halotolerans (e.g., NRIC 1627 strain and the like).

Examples of the microorganism belonging to the genus Akkermansia include a microorganism belonging to Akkermansia muciniphila (e.g., DSM 22959 strain, DSM 26127 strain, and the like).

Examples of the microorganism belonging to the genus Anaerofustis include a microorganism belonging to Anaerofustis stercorihominis (e.g., DSM 17244 strain and the like).

Examples of the microorganism belonging to the genus Anaerotruncus include a microorganism belonging to Anaerotruncus colihominis (e.g., DSM 17241 strain and the like).

Examples of the microorganism belonging to the genus Arcobacter include a microorganism belonging to Arcobacter butzleri (e.g., DSM 107942 strain and the like).

Examples of the microorganism belonging to the genus Bacteroides include a microorganism belonging to Bacteroides faecichinchillae (e.g., DSM 26883 strain and the like), and a microorganism belonging to Bacteroides stercoris (e.g., DSM 19555 strain and the like).

Examples of the microorganism belonging to the genus Clostridium include a microorganism belonging to Clostridium bolteae (e.g., NITE BP-03384 strain, NITE BP-03383 strain, and the like), a microorganism belonging to Clostridium celerecresens (e.g., JCM 15734 strain and the like), a microorganism belonging to Clostridium clostridioforme (e.g., JCM 1291 strain and the like), a microorganism belonging to Clostridium hathewayi (e.g., DSM 13479 strain, DSM 13480 strain, and the like), a microorganism belonging to Clostridium paraptrificum (e.g., JCM 1293 strain and the like), a microorganism belonging to Clostridium ramosum (e.g., JCM 1298 strain and the like), and a microorganism belonging to Clostridium sporogenes (e.g., JCM 1416 strain and the like).

Examples of the microorganism belonging to the genus Coprobacillus include a microorganism belonging to Coprobacillus cateniformis (e.g., DSM 15921 strain and the like).

Examples of the microorganism belonging to the genus Dielma include a microorganism belonging to Dielma fastidiosa (e.g., DSM 26099 strain and the like).

Examples of the microorganism belonging to the genus Escherichia include a microorganism belonging to Escherichia coli (e.g., ATCC 27325 strain and the like), a microorganism belonging to Escherichia fergusonii (e.g., NITE BP-03390 strain. NITE BP-03389 strain, NITE BP-03388 strain, and the like).

Examples of the microorganism belonging to the genus Eubacterium include a microorganism belonging to Eubacterium limosum (e.g., JCM 6501 strain and the like), and a microorganism belonging to Eubacterium ramulus (e.g., DSM 16296 strain and the like).

Examples of the microorganism belonging to the genus Faecalicoccus include a microorganism belonging to Faecalicoccus pleomorphus (e.g., NITE BP-03392 strain, NITE BP-03393 strain, NITE BP-03391 strain, and the like), and Faecalicoccus sp. NITE BP-03394 strain.

Examples of the microorganism belonging to the genus Finegoldia include a microorganism belonging to Finegoldia magna (e.g., JCM 1766 strain and the like).

Examples of the microorganism belonging to the genus Hungatella include a microorganism belonging to Hungatella effluvii (e.g., DSM 24995 strain and the like), a microorganism belonging to the genus Hungatella hathewayi (e.g., NITE BP-03396 strain, NITE BP-03395 strain, and the like), Hungatella sp. NITE BP-03398 strain, and Hungatella sp. NITE BP-03385 strain.

Examples of the microorganism belonging to the genus Intestinimonas include a microorganism belonging to Intestinimonas butyriciproducens (e.g., NITE BP-03399 strain, NITE BP-03397 strain, and the like).

Examples of the microorganism belonging to the genus Parascardovia include a microorganism belonging to Parascardovia denticolens (e.g., JCM 12538 strain and the like).

Examples of the microorganism belonging to the genus Prevotella include a microorganism belonging to Prevotella rara (e.g., DSM 105141 strain and the like), and a microorganism belonging to Prevotella melaninogenica (e.g., JCM 6325 strain and the like).

Examples of the microorganism belonging to the genus Solobacterium include a microorganism belonging to Solobacterium moorei (e.g., DSM 22971 strain and the like).

Examples of the microorganism belonging to the genus Sutterella include a microorganism belonging to Sutterella megalosphaeroides (e.g., DSM 106861 strain and the like).

Examples of the microorganism belonging to the genus Bifidobacterium include a microorganism belonging to Bifidobacterium actinocoloniiforme (e.g., JCM 18048 strain and the like), a microorganism belonging to Bifidobacterium adolescentis (e.g., JCM 1275 strain and the like), a microorganism belonging to Bifidobacterium animalis subsp. animalis (e.g., JCM 1190 strain and the like), a microorganism belonging to Bifidobacterium bifidum (e.g., JCM 1255 strain and the like), a microorganism belonging to Bifidobacterium callitrichos (e.g., JCM 17296 strain and the like), a microorganism belonging to Bifidobacterium coryneforme (e.g., JCM 5819 strain and the like), a microorganism belonging to Bifidobacterium gallinarum (e.g., JCM 6291 strain and the like), a microorganism belonging to Bifidobacterium indicum (e.g., JCM 1302 strain and the like), a microorganism belonging to Bifidobacterium longum subsp. longum (e.g., JCM 1217 strain and the like), a microorganism belonging to Bifidobacterium longum subsp. suis (e.g., JCM 1269 strain and the like), a microorganism belonging to Bifidobacterium magnum (e.g., JCM 1218 strain and the like), a microorganism belonging to Bifidobacterium psychraerophilum (e.g., JCM 15958 strain and the like), a microorganism belonging to Bifidobacterium pullorum (e.g., JCM 1214 strain and the like), a microorganism belonging to Bifidobacterium reuteri (e.g., JCM 17295 strain and the like), a microorganism belonging to Bifidobacterium ruminantium (e.g., JCM 8222 strain and the like), a microorganism belonging to Bifidobacterium saeculare (e.g., JCM 8223 strain and the like), a microorganism belonging to Bifidobacterium scardovii (e.g., JCM 12489 strain and the like), a microorganism belonging to Bifidobacterium stellenboschense (e.g., JCM 17298 strain and the like), a microorganism belonging to Bifidobacterium thermacidophilum subsp. thermacidophilum (e.g., JCM 11165 strain and the like), a microorganism belonging to Bifidobacterium catenulatum subsp. catenulatum (e.g., DSM 16992 strain and the like), a microorganism belonging to Bifidobacterium catulorum (e.g., DSM 103154 strain and the like), a microorganism belonging to Bifidobacterium jacchi (e.g., DSM 103362 strain and the like), a microorganism belonging to Bifidobacterium primatium (e.g., DSM 100687 strain and the like), and a microorganism belonging to Bifidobacterium simiarum (e.g., DSM 103153 strain and the like).

Examples of the microorganism belonging to the genus Anaerostipes include a microorganism belonging to Anaerostipes caccae (e.g., JCM 13470 strain and the like).

Examples of the microorganism belonging to the genus Chitinophaga include a microorganism belonging to Chitinophaga skermanii (e.g., NBRC 109753 strain and the like).

Examples of the microorganism belonging to the genus Citrobacter include a microorganism belonging to Citrobacter sediakii (e.g., NBRC 105722 strain and the like).

Examples of the microorganism belonging to the genus Clostridioides include a microorganism belonging to Clostridioides difficile (e.g., JCM 1296 strain and the like).

Examples of the microorganism belonging to the genus Cryptobacterium include a microorganism belonging to Cryptobacterium sp.

NITE BP-03476 strain.

Examples of the microorganism belonging to the genus Edwardsiella include a microorganism belonging to Edwardsiella tarda (e.g., NBRC 105688 strain and the like).

Examples of the microorganism belonging to the genus Klebsiella include a microorganism belonging to Klebsiella aerogenes (e.g., DSM 30053 strain and the like).

Examples of the microorganism belonging to the genus Lacrimispora include a microorganism belonging to Lacrimispora sphenoides (e.g., JCM 1415 strain and the like).

Examples of the microorganism belonging to the genus Megasphaera include a microorganism belonging to Megasphaera elsdenii (e.g., JCM 1772 strain and the like).

Examples of the microorganism belonging to the genus Parabacteroides include a microorganism belonging to Parabacteroides distasonis (e.g., JCM 5825 strain and the like).

Examples of the microorganism belonging to the genus Providencia include a microorganism belonging to Providencia alcalifaciens (e.g., NBRC 105687 strain and the like).

Examples of the microorganism belonging to the genus Ruminococcus include a microorganism belonging to Ruminococcus gnavus (e.g., JCM 6515 strain and the like).

Examples of the microorganism belonging to the genus Yersinia include a microorganism belonging to Yersinia bercovieri (e.g., NBRC 105717 strain and the like), and a microorganism belonging to Yersinia rohdei (e.g., NBRC 105715 strain and the like).

The Clostridium bolteae NITE BP-03383 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03383 as of Feb. 16, 2021, under the Budapest Treaty.

The Clostridium bolteae NITE BP-03384 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03384 as of Feb. 16, 2021, under the Budapest Treaty.

The Hungatella sp. NITE BP-03385 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03385 as of Feb. 16, 2021, under the Budapest Treaty.

The Enterococcus avium NITE BP-03386 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03386 as of Feb. 16, 2021, under the Budapest Treaty.

The Enterococcus avium NITE BP-03387 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03387 as of Feb. 16, 2021, under the Budapest Treaty.

The Escherichia fergusonii NITE BP-03388 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03388 as of Feb. 16, 2021, under the Budapest Treaty.

The Escherichia fergusonii NITE BP-03389 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03389 as of Feb. 16, 2021, under the Budapest Treaty.

The Escherichia fergusonii NITE BP-03390 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03390 as of Feb. 16, 2021, under the Budapest Treaty.

The Faecalicoccus pleomorphus NITE BP-03391 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03391 as of Feb. 16, 2021, under the Budapest Treaty.

The Faecalicoccus pleomorphus NITE BP-03392 strain was internationally deposited with Patent Microorganisms Depositary. National Institute of Technology and Evaluation (zip code: 292-0818, address: #122, 2-5-8 Kazusa Kamatari, Kisarazu-shi, Chiba) under accession No. NITE BP-03392 as of Feb. 16, 2021, under the Budapest Treaty.

The Faecalicoccus pleomorphus NITE BP-03393 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03393 as of Feb. 16, 2021, under the Budapest Treaty.

The Faecalicoccus pleomorphus NITE BP-03394 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03394 as of Feb. 16, 2021, under the Budapest Treaty.

The Hungatella hathewayi NITE BP-03395 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03395 as of Feb. 16, 2021, under the Budapest Treaty.

The Hungatella hathewayi NITE BP-03396 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03396 as of Feb. 16, 2021, under the Budapest Treaty.

The Intestinimonas butyriciproducens NITE BP-03397 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03397 as of Feb. 16, 2021, under the Budapest Treaty.

The Hungatella sp. NITE BP-03398 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03398 as of Feb. 16, 2021, under the Budapest Treaty.

The Intestinimonas butyriciproducens NITE BP-03399 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03399 as of Feb. 16, 2021, under the Budapest Treaty.

The Cryptobacterium sp. NITE BP-03476 strain was internationally deposited with Patent Microorganisms Depositary, National Institute of Technology and Evaluation (zip code: 292-0818, address: #122, 2-5-8 Kazusa Kamatari, Kisarazu-shi, Chiba) under accession No. NITE BP-03476 as of May 7, 2021, under the Budapest Treaty.

The Carnobacterium divergens NBRC 15683 strain will be described as an example. In the present aspect, the Carnobacterium divergens NBRC 15683 strain is not limited to the deposited strain, and may be a strain substantially equivalent to the deposited strain. The strain substantially equivalent refers to a strain belonging to the same genus or species as the deposited strain and having an ability of promoting demethylation of a microorganism having a demethylation ability of eliminating a methyl group(s) of a methoxy group(s) from a compound with the methoxy group(s) in a side chain(s). In addition. the strain substantially equivalent is a strain in which the base sequence of the 16S rRNA gene is 97% or greater, preferably 98.5% or greater, more preferably 98.7% or greater, even more preferably 99% or greater, and even more preferably 100% homologous to the base sequence of the 16S rRNA gene of the deposited strain. Furthermore, the deposited strain may be a strain that is grown from the deposited strain or the strain substantially equivalent thereto, by mutation treatment, genetic recombination, selection of a natural mutant strain, or the like, as long as the strain has an ability of promoting demethylation of a microorganism having a demethylation ability of eliminating a methyl group(s) of a methoxy group(s) from a compound with the methoxy group(s) in a side chain(s).

This also applies to the other deposited strains described above.

In the present aspect, one kind or two or more kinds of the microorganisms may be used, and one strain or two or more strains may be used.

Resting Body of Microorganism Having Activity to Promote Demethylation

In the present aspect, the microorganism having the activity to promote demethylation includes a resting body thereof.

The “resting body” refers to a microorganism body obtained by removing culture medium components from a cultured microorganism by manipulations such as centrifugation, washing the microorganism with a salt solution or a buffer solution, and suspending the microorganism in the same liquid as the washing solution, the microorganism body being in a non-proliferative state. In the present aspect, the resting body refers to at least a microorganism body having a metabolic system that can promote demethylation of a microorganism having a demethylation ability of eliminating a methyl group(s) of a methoxy group(s) from a compound with the methoxy group(s) in a side chain(s). In a case where the microorganism is a bacterium having an activity to promote demethylation, the resting body is a resting bacterial body.

Examples of the salt solution include physiological saline. Examples of the buffer solution include a phosphate buffer solution, a tris-hydrochloric acid buffer solution, a citrate-phosphate buffer solution, a citrate buffer solution, a MOPS buffer solution, an acetate buffer solution, and a glycine buffer solution. In any case, the pH and concentration can be appropriately adjusted in accordance with a known method.

The microorganism having the activity to promote demethylation in the present aspect is preferably a microorganism having an activity to promote regeneration of tetrahydrofolic acid (THF) from 5-methyltetrahydrofolate (5-CH₃-THF). Details of the preferred aspect are as follows.

When the microorganism having a demethylation ability of eliminating a methyl group(s) of a methoxy group(s) from the compound with the methoxy group(s) in a side chain(s) eliminates the methyl group(s) of the methoxy group(s) of the compound with the methoxy group(s) in the side chain(s) to produce the demethylated compound, 5-methyltetrahydrofolate (5-CH₃-THF) is produced from tetrahydrofolic acid (THF) via a conjugated system in the microorganism having a demethylation ability. 5-CH₃-THF is converted to 5,10-methylenetetrahydrofolate (5,10-CH₂-THF) by methylenetetrahydrofolate reductase (MTHFR) in the microorganism having a demethylation ability.

On the other hand, the microorganism having an activity to promote demethylation (the microorganism having an activity to promote regeneration of tetrahydrofolic acid (THF) from 5-methyltetrahydrofolate (5-CH₃-THF) in the preferred aspect) is preferably a microorganism producing dihydrofolate reductase-thymidylate synthase (DHFR-TS (thyA)).

The produced DHFR-TS (thyA) is incorporated into the microorganism having a demethylation ability. As a result, in the microorganism having a demethylation ability, 5,10-CH₂-THF is converted to dihydrofolate (DHF) by DHFR-TS (thyA). In the microorganism having a demethylation ability. DHF is converted into THF (i.e., THF is regenerated) by dihydrofolate reductase (DHFR) of the microorganism having a demethylation ability. With this mechanism, production of the demethylated compound is promoted by eliminating the methyl group(s) of the methoxy group(s) from the compound with the methoxy group(s) in the side chain(s). At the time, the dihydrofolate reductase-thymidylate synthase (DHFR-TS (thyA)) is preferably dihydrofolate reductase-thymidylate synthase (DHFR-TS (thyA)) (E.C.1. 5.1.3).

In addition to or separately from this, the microorganism having an activity to promote demethylation (the microorganism having an activity to promote regeneration of tetrahydrofolic acid (THF) from 5-methyltetrahydrofolate (5-CH₃-THF) in the preferred aspect) is preferably a microorganism producing glycine hydroxymethyltransferase (SHMT (glyA)). The produced (SHMT (glyA)) is incorporated into the microorganism having a demethylation ability. As a result, in the microorganism having a demethylation ability, 5,10-CH₂-THF is converted to THF (i.e., THF is regenerated) by SHMT (glyA). With this mechanism, production of the demethylated compound is promoted by eliminating of the methyl group(s) of the methoxy group(s) from the compound with the methoxy group(s) in the side chain(s). At the time, the glycine hydroxymethyltransferase (SHMT (glyA)) is preferably glycine hydroxymethyltransferase (SHMT (glyA)) (E.C.2.1.2.1).

Accordingly, the microorganism having an activity to promote demethylation (the microorganism having an activity to promote regeneration of tetrahydrofolic acid (THF) from 5-methyltetrahydrofolate (5-CH₃-THF) in the preferred aspect) is preferably a microorganism producing dihydrofolate reductase-thymidylate synthase (DHFR-TS (thyA)) and/or glycine hydroxymethyltransferase (SHMT (glyA)).

The dihydrofolate reductase-thymidylate synthase (DHFR-TS (thyA)) is more preferably dihydrofolate reductase-thymidylate synthase (DHFR-TS (thyA)) (E.C.1.5.1.3). In addition, the glycine hydroxymethyltransferase (SHMT (glyA)) is more preferably glycine hydroxymethyltransferase (SHMT (glyA)) (E.C.2.1.2. 1).

Examples of the microorganism producing such an enzyme include the microorganism exemplified as the microorganism having an activity to promote demethylation.

Solution Containing Compound with Methoxy Group in Side Chain

The solution containing a compound with a methoxy group(s) in a side chain(s) in the present aspect is not particularly limited as long as in the solution, the microorganism having a demethylation ability of eliminating a methyl group(s) of a methoxy group(s) from a compound with the methoxy group(s) in a side chain(s) can eliminate a methyl group(s) of a methoxy group(s) of the compound with the methoxy group(s) in a side chain(s) to produce a demethylated compound and the microorganism having an activity to promote demethylation can promote the elimination of the methyl group(s) of the methoxy group(s) from the compound with the methoxy group(s) in the side chain(s). The solution is preferably a culture medium, and more preferably a culture medium described in “Culture Medium and Production of Demethylated Compound by Culture Medium” described below. In addition, in a case where the microorganism is a resting body, the solution is preferably the salt solution or buffer solution described above.

Note that in any case, the “culture medium” as described herein refers to a solution in which the microorganism can grow, including minimal media, and does not include a solution in which the microorganism cannot grow, for example, the salt solution or buffer solution described above.

In a case where the compound with a methoxy group(s) in a side chain(s) is added to the solution, it may be added before or during production of the demethylated compound, and may be added all at once, sequentially or continuously.

The content in the solution of the compound with a methoxy group(s) in a side chain(s) is preferably 0.001 g/L or more, more preferably 0.01 g/L or more, even more preferably 0.1 g/L or more, and still more preferably 1 g/L or more. On the other hand, the content thereof is ordinarily not greater than 100 g/L, preferably not greater than 20 g/L, and more preferably not greater than 10 g/L.

Culture Medium and Production of Demethylated Compound by Culture Medium

In the above step, the solution is preferably a culture medium. The culture medium is not particularly limited, and for example, ANAEROBE BASAL BROTH (ABB culture medium) available from Oxoid Ltd., Wilkins-Chalgren Anaerobe Broth (CM0643) available from Oxoid Ltd., and a GAM culture medium and a modified GAM culture medium available from Nissui Pharmaceutical Co., Ltd. can be used.

Furthermore, a water soluble organic material can be added, as a carbon source, to the culture medium. Examples of the water soluble organic material include the following compounds: sugars such as glucose, arabinose, sorbitol. fructose, mannose, sucrose, trehalose, and xylose; alcohols such as glycerol; and organic acids such as valeric acid, butyric acid, propionic acid, acetic acid, formic acid, and fumaric acid.

The concentration of organic material added in the culture medium as a carbon source can be adjusted, as appropriate, for efficient growth. In general, the addition amount is selected from the range of 0.1 to 10 wt/vol %.

In addition to the carbon source described above, a nitrogen source can be added to the culture medium. Various nitrogen compounds that may be used ordinarily in fermentation can be used as the nitrogen source.

Preferred examples of inorganic nitrogen sources include ammonium salts and nitrates, and more preferred examples thereof include ammonium sulfate, ammonium chloride, ammonium phosphate, ammonium hydrogenphosphate, potassium nitrate, and sodium nitrate.

On the other hand, examples of organic nitrogen sources include amino acids, yeast extracts, peptones (e.g., polypeptone N, soy peptone), meat extracts (e.g., Ehrlich Bonito Extract, Lab Lemco powder, bouillon), seafood extracts, liver extracts, digested serum powder, and fish oil.

In addition to the carbon source and the nitrogen source, in some cases, growth and activity can be enhanced by adding cofactors such as vitamins or inorganic compounds such as various salts to the culture medium. Examples of animal- and plant-derived cofactors for microbial growth. such as inorganic compounds. vitamins, and fatty acids, include the following.

| Inorganic Compounds | Vitamins |
|---|---|
| Potassium dihydrogen phosphate | Biotin |
| Magnesium sulfate | Folic acid |
| Manganese sulfate | Pyridoxine |
| Sodium chloride | Thiamine |
| Cobalt chloride | Riboflavin |
| Calcium chloride | Nicotinic acid |
| Zinc sulfate | Pantothenic acid |
| Copper sulfate | Vitamin B12 |
| Alum | Thioctic acid |
| Sodium molybdate | p-Aminobenzoic acid |
| Potassium chloride | Vitamin K |
| Boric acid and the like | |
| Nickel chloride | |
| Sodium tungstate | |
| Sodium selenate | |
| Iron (II) ammonium sulfate | |
| Sodium acetate trihydrate | |
| Magnesium sulfate heptahydrate | |
| Manganese sulfate tetrahydrate | |

In addition, it is preferable to add a reducing agent such as cysteine, cystine, sodium sulfide, sulfite, ascorbic acid, glutathione, thioglycolic acid, or rutin, or an enzyme capable of decomposing an active oxygen species such as catalase or superoxide mutase to the culture medium because the growth may be improved.

The gas phase and aqueous phase during culturing preferably do not contain air or oxygen, and examples include gas and aqueous phases that contain nitrogen and/or hydrogen in any ratio, and gas and aqueous phases that contain nitrogen and/or carbon dioxide in any ratio, and the gas phase and aqueous phase preferably contain hydrogen. The proportion of hydrogen in the gas phase is usually 0.5 vol % or greater, preferably 1.0 vol % or greater, and more preferably 2.0 vol % or greater, but usually 100 vol % or less, preferably 20 vol % or less, and more preferably 10 vol % or less because production of a demethylated compound is promoted and/or the promotion of demethylation is further promoted.

The method of establishing such an environment for the gas phase and aqueous phase in the culture medium is not particularly limited, and methods such as, for example, replacing the gas phase with the above-mentioned gas prior to culture, additionally during culture supplying the gas from the bottom of the incubator and/or supplying the gas to the gas phase part of the incubator, and bubbling the aqueous phase with the above-mentioned gas prior to culture can be adopted. Hydrogen gas may be used as it is as the hydrogen. Furthermore, a hydrogen raw material such as formic acid and/or a salt thereof may be added to the culture medium to produce hydrogen during culture by an action of the microorganism.

An aeration amount is preferably 0.005 to 2 vvm, and more preferably 0.05 to 0.5 vvm. The mixed gas can also be supplied as nanobubbles.

The culture temperature is preferably from 20° C. to 45° C., more preferably from 25° C. to 40° C., and even more preferably from 30° C. to 37° C.

The pressurization condition of the incubator is not particularly limited as long as it is a condition that allows growth, but is preferably in the range of 0.001 to 1 MPa, and more preferably 0.01 to 0.5 MPa.

The culture time is preferably from 8 to 340 hours, more preferably from 12 to 170 hours, and even more preferably from 16 to 120 hours.

In addition, it is preferable to add a surfactant, an adsorbent, a clathrate compound, or the like to the culture solution because production of the demethylated compound may be promoted and/or promotion of the demethylation may be further promoted.

Examples of the surfactant include Tween 80, and can be added at an amount of approximately from 0.001 g/L to 10 g/L.

Examples of the adsorbent include cellulose and derivatives thereof; dextrin; hydrophobic adsorbents Diaion HP series and Sepabeades series, available from Mitsubishi Chemical Corporation; and Amberlite XAD series, available from Organo Corporation.

Examples of the clathrate compound include α-cyclodextrin, β-cyclodextrin, γ-cyclodextrin, and cluster dextrins (highly branched cyclic dextrins), and may include analogs thereof. Examples of the analogs include methyl-β-cyclodextrin, trimethyl-β-cyclodextrin, and hydroxypropyl-β-cyclodextrin. Among these, γ-cyclodextrin may be the most effective and is thus preferred in such cases. Furthermore, coexistence of two or more clathrate compounds is preferable because production of the demethylated compound may be further promoted and/or promotion of the demethylation may be further promoted.

In terms of a total molar ratio of the clathrate compounds when the total amount of the compound with a methoxy group(s) in a side chain(s) is 1, the addition amount of the clathrate compound is usually 0.1 or greater, preferably 0.5 or greater, and more preferably 1.0 or greater, and usually 5.0 or less, preferably 2.5 or less, and more preferably 2.0 or less.

The content of the microorganism having a demethylating ability of eliminating a methyl group(s) of a methoxy group(s) from the compound with the methoxy group(s) in a side chain(s) in the solution comprising the compound with a methoxy group(s) in a side chain(s) is not particularly limited as long as the microorganism having a demethylation ability of eliminating a methyl group(s) of a methoxy group(s) from the compound with the methoxy group(s) in a side chain(s) can eliminate a methyl group(s) of a methoxy group(s) from the compound with the methoxy group(s) in a side chain(s) to produce a demethylated compound, and the microorganism having an activity to promote demethylation promotes the elimination of the methyl group(s) of the methoxy group(s) from the compound with the methoxy group(s) in a side chain(s).

The content of the microorganism having an activity to promote demethylation in the solution comprising a compound with a methoxy group(s) in a side chain(s) is not particularly limited as long as the microorganism having a demethylation ability of eliminating a methyl group(s) of a methoxy group(s) from a compound with the methoxy group(s) in a side chain(s) can eliminate the methyl group(s) of the methoxy group(s) of the compound with the methoxy group(s) in a side chain(s) to produce a demethylated compound and the microorganism having an activity to promote demethylation promotes the elimination of the methyl group(s) of the methoxy group(s) from the compound with the methoxy group(s) in a side chain(s).

Production of Demethylated Compound by Resting Body

In a case where the microorganism is a resting body, the solution is preferably a salt solution or a buffer solution described in the section “Resting Body of Microorganism Having Demethylation Ability of Eliminating Methyl Group of Methoxy Group from Compound with Methoxy Group in Side chain” instead of the culture medium.

For the other conditions, the description in the section “Culture Medium and Production of Demethylated Compound by Culture Medium” is referred to.

Other Steps

The present aspect may include, for example, a step of quantitatively determining the resulting demethylated compound. The method can be in accordance with routine methods. For example, a part of the culture solution is collected, appropriately diluted, and stirred well, and the obtained solution is then filtered using a membrane such as a polytetrafluoroethylene (PTFE) membrane to remove insoluble matter, and the resulting product can then be quantified using high performance liquid chromatography.

The present aspect may also include a step of recovering the resulting demethylated compound. The recovery step includes steps such as a purification step and a concentration step. As a purification treatment in the purification step, treatments such as sterilization of the microorganism through heat or the like; bacteria elimination through a method such as microfiltration (MF) or ultrafiltration (UF); removal of solids and polymeric substances; extraction using an organic solvent, an ionic liquid, or the like; and adsorption and decolorization using a hydrophobic adsorbent, an ion exchange resin, an activated carbon column, or the like can be implemented. Furthermore, examples of a concentration treatment in the concentration step include concentration using an evaporator, reverse osmosis membrane, or the like.

In addition, a solution containing the resulting demethylated compound can be formed into a powder through freeze drying, spray drying, or the like. In the formation of a powder, an excipient such as lactose, dextrin, or corn starch can be added.

Composition for Promoting Production of Demethylated Compound

Another aspect of the present disclosure is a composition for promoting production of a demethylated compound in which a methyl group(s) of a methoxy group(s) has eliminated from a compound with the methoxy group(s) in a side chain(s), comprising a microorganism having a demethylation ability of eliminating a methyl group(s) of a methoxy group(s) from the compound with the methoxy group(s) in the side chain(s), and a microorganism having an activity to promote the demethylation.

For details of the microorganism having a demethylation ability of eliminating a methyl group(s) of a methoxy group(s) from a compound with the methoxy group(s) in a side chain(s) in the present aspect, the above description is referred to.

For details of the microorganism having an activity to promote the demethylation in the present aspect, the above description is referred to.

The composition according to the present aspect may comprise a component other than the two microorganisms as long as the microorganism having a demethylation ability of eliminating a methyl group(s) of a methoxy group(s) from a compound with the methoxy group(s) in a side chain(s) can eliminate a methyl group(s) of a methoxy group(s) from a compound with the methoxy group(s) in a side chain(s) to produce a demethylated compound and the microorganism having an activity to promote demethylation promotes the elimination of the methyl group(s) of the methoxy group(s) from the compound with the methoxy group(s) in the side chain(s).

The content of the microorganism having a demethylation ability of eliminating a methyl group(s) of a methoxy group(s) from a compound with the methoxy group(s) in a side chain(s) relative to the total amount of the composition according to the present aspect is not particularly limited as long as the microorganism having a demethylation ability of eliminating a methyl group(s) of a methoxy group(s) from a compound with the methoxy group(s) in a side chain(s) can eliminate a methyl group(s) of a methoxy group(s) of a compound with the methoxy group(s) in a side chain(s) to produce a demethylated compound and the microorganism having an activity to promote demethylation promotes the elimination of the methyl group(s) of the methoxy group(s) from the compound with the methoxy group(s) in the side chain(s).

The content of the microorganism having an activity to promote the demethylation relative to the total amount of the composition according to the present aspect is not particularly limited as long as the microorganism having a demethylation ability of eliminating a methyl group(s) of a methoxy group(s) from a compound with the methoxy group(s) in a side chain(s) can eliminate a methyl group(s) of a methoxy group(s) of a compound with the methoxy group(s) in a side chain(s) to produce a demethylated compound and the microorganism having an activity to promote demethylation promotes the elimination of the methyl group(s) of the methoxy group(s) from the compound with the methoxy group(s) in the side chain(s).

The composition according to the present aspect is not particularly limited as long as the microorganism having a demethylation ability of eliminating a methyl group(s) of a methoxy group(s) from a compound with the methoxy group(s) in a side chain(s) can eliminate a methyl group(s) of a methoxy group(s) of a compound with the methoxy group(s) in a side chain(s) to produce a demethylated compound and the microorganism having an activity to promote demethylation promotes the elimination of the methyl group(s) of the methoxy group(s) from the compound with the methoxy group(s) in the side chain(s). Examples of the composition include the solution containing a compound having a methoxy group(s) in a side chain(s) in the above aspect, and containing the microorganism having a demethylation ability of eliminating a methyl group(s) of a methoxy group(s) from a compound with the methoxy group(s) in a side chain(s), and the microorganism having an activity to promote the demethylation.

For details of the solution containing a compound with a methoxy group(s) in a side chain(s), the description of the “Solution Containing Compound with Methoxy Group in Side Chain” in the above aspect is referred to.

### EXAMPLES

Examples will be described below, but none of the examples shall be construed as limiting the present disclosure.

Note that in Examples, a microorganism having a demethylation ability of eliminating a methyl group(s) of a methoxy group(s) from a compound with the methoxy group(s) in a side chain(s) may be referred to as a “first microorganism”, and a microorganism having an activity to promote the demethylation may be referred to as a “second microorganism”

Method for Producing 8-Prenylnaringenin from Isoxanthohumol (1)

### Comparative Example 1

After isoxanthohumol (final concentration of 50 mg/L) was added to a modified GAM medium (available from Nissui Pharma Medical Sales Co Ltd), the mixture was heated and sterilized, the gas phase was replaced with a gas of N₂:CO₂:H₂ (80%:10%:10%), and the resulting product was used as a fermentation medium. Blautia sp. DC 3652 (NITE BP-02924) strain was inoculated as a first microorganism to the fermentation medium and subjected to anaerobic culture at 37° C. for 72 hours. After the culture, 5 mL of the culture solution was diluted in 3 times its volume of ethanol and filtered through a 0.45 μm filter, and the filtrate was then subjected to quantitative analysis of 8-prenylnaringenin by HPLC under the following conditions. No second microorganism was used.

HPLC Conditions
Column: Inertsil ODS-3 (4.6 mm×25 cm, 5 μm)
Eluent A: H₂O/Formic acid (99/1)
Eluent B: Acetonitrile/Formic acid (99/1)
Gradient condition: Solution B; 0 to 5 min (20%), then 10 min (70%), and then 30 min (70%)
Column temperature: 40° C.
Flow rate: 1.0 mL/min
Detection: UV at 290 nm

### Example 1

The analysis was performed in the same manner as in Comparative Example 1 except that in addition to the first microorganism, microorganisms described in Table 1-1 to Table 1-8 were inoculated as a second microorganism into the fermentation medium.

Conversion rates from isoxanthohumol to 8-prenylnaringenin are shown in Table 1-1 to Table 1-8. No. 1 in the table corresponds to Comparative Example 1.

In the tables, “*1” represents the first microorganism, “*2” represents the second microorganism, and “*3” represents the conversion rates from isoxanthohumol to 8-prenylnaringenin.

TABLE 1-1

| No. | * 1 | * 2 | * 3 |
|---|---|---|---|
| 1 | Blautia sp. DC 3652 | — | 10.5% |
| 2 | Blautia sp. DC 3652 | Weissella confusa DSM 20196 | 92.3% |
| 3 | Blautia sp. DC 3652 | Streptococcus uberis NRIC 1153 | 78.2% |
| 4 | Blautia sp. DC 3652 | Lactobacillus graminis JCM 9503 | 77.8% |
| 5 | Blautia sp. DC 3652 | Lactobacillus plantarum NCIMB 8826 | 77.5% |
| 6 | Blautia sp. DC 3652 | Lactobacillus rhamnosus DSM 20021 | 77.3% |
| 7 | Blautia sp. DC 3652 | Lactobacillus paralimentarius | 75.6% |
| | | NBRC 107149 | |
| 8 | Blautia sp. DC 3652 | Lactobacillus saerimneri | 75.4% |
| | | NBRC 107826 | |
| 9 | Blautia sp. DC 3652 | Lactobacillus delbrueckii subsp. | 74.9% |
| | | delbrueckii IAM 1928 | |
| 10 | Blautia sp. DC 3652 | Lactobacillus brevis NRIC 1037 | 74.8% |
| 11 | Blautia sp. DC 3652 | Lactobacillus casei AHU 1055 | 74.6% |
| 12 | Blautia sp. DC 3652 | Lactobacillus leichmannii AHU 1681 | 73.7% |
| 13 | Blautia sp. DC 3652 | Lactobacillus hilgardii DSM 20051 | 73.2% |
| 14 | Blautia sp. DC 3652 | Carnobacterium divergens | 72.9% |
| | | NBRC 15683 | |
| 15 | Blautia sp. DC 3652 | Lactobacillus pentosus NBRC 106467 | 72.3% |
| 16 | Blautia sp. DC 3652 | Leuconostoc lactis IFO 12455 | 71.0% |
| 17 | Blautia sp. DC 3652 | Streptococcus equinus NRIC 1139 | 70.8% |
| 18 | Blautia sp. DC 3652 | Lactobacillus fabifermentans | 70.6% |
| | | DSM 21115 | |
| 19 | Blautia sp. DC 3652 | Lactobacillus pobuzihii JCM 18084 | 70.4% |
| 20 | Blautia sp. DC 3652 | Lactobacillus antri JCM 15950 | 69.5% |
| 21 | Blautia sp. DC 3652 | Lactobacillus xiangfangensis | 69.4% |
| | | NBRC 108914 | |
| 22 | Blautia sp. DC 3652 | Lactobacillus songhuajiangensis | 68.9% |
| | | JCM 30918 | |
| 23 | Blautia sp. DC 3652 | Lactobacillus harbinensis | 68.4% |
| | | NBRC 100982 | |
| 24 | Blautia sp. DC 3652 | Leuconostoc dextranicum IFO 3347 | 68.2% |
| 25 | Blautia sp. DC 3652 | Leuconostoc mesenteroides subsp. | 68.1% |
| | | cremoris IAM 1087 | |
| 26 | Blautia sp. DC 3652 | Lactobacillus siliginis NBRC 101315 | 68.1% |
| 27 | Blautia sp. DC 3652 | Lactobacillus lactis AHU 1059 | 68.0% |
| 28 | Blautia sp. DC 3652 | Lactobacillus paraplantarum | 68.0% |
| | | NBRC 107151 | |
| 29 | Blautia sp. DC 3652 | Lactobacillus cryzae JCM 18671 | 67.4% |
| 30 | Blautia sp. DC 3652 | Lactobacillus planterum subsp. | 66.8% |
| | | argentoratensis NBRC 106468 | |
| 31 | Blautia sp. DC 3652 | Lactobacillus mixtipabuli JCM 19805 | 66.6% |
| 32 | Blautia sp. DC 3652 | Lactobacillus buchneri NRIC 1040 | 66.3% |
| 33 | Blautia sp. DC 3652 | Lactobacillus rossiae JCM 16176 | 66.2% |
| 34 | Blautia sp. DC 3652 | Lactobacillus zymae NBRC 107157 | 66.0% |
| 35 | Blautia sp. DC 3652 | Lactobacillus fructivorans | 65.7% |
| | | NBRC 13954 | |
| 36 | Blautia sp. DC 3652 | Lactobacillus acidifarinae | 65.6% |
| | | NBRC 107156 | |
| 37 | Blautia sp. DC 3652 | Lactobacillus ceni JCM 18036 | 65.4% |
| 38 | Blautia sp. DC 3652 | Lactobacillus composti JCM 14202 | 65.2% |
| 39 | Blautia sp. DC 3652 | Lactobacillus parabrevis | 65.2% |
| | | NBRC 107154 | |
| 40 | Blautia sp. DC 3652 | Lactobacillus oris JCM 11028 | 64.3% |

TABLE 1-2

| No. | * 1 | * 2 | * 3 |
|---|---|---|---|
| 41 | Blautia sp. DC 3652 | Lactobacillus delbrueckii AHU 1056 | 64.3% |
| 42 | Blautia sp. DC 3652 | Lactobacillus capillatus JCM 15044 | 64.0% |
| 43 | Blautia sp. DC 3652 | Leuconostoc citreum JCM 9698 | 63.4% |
| 44 | Blautia sp. DC 3652 | Lactobacillus pantheris | 63.3% |
| | | NBRC 106106 | |
| 45 | Blautia sp. DC 3652 | Lactobacillus vaccinostercus | 62.9% |
| | | NRIC 1075 | |
| 46 | Blautia sp. DC 3652 | Lactobacillus perolens JCM 12534 | 62.7% |
| 47 | Blautia sp. DC 3652 | Lactobacillus zeae DSM 20178 | 62.4% |
| 48 | Blautia sp. DC 3652 | Lactobacillus tucceti JCM 18037 | 62.3% |
| 49 | Blautia sp. DC 3652 | Lactobacillus sunkii JCM 15039 | 62.3% |
| 50 | Blautia sp. DC 3652 | Pediococcus argentinicus JCM 30771 | 61.7% |
| 51 | Blautia sp. DC 3652 | Lactobacillus kefiri NRIC 1693 | 61.7% |
| 52 | Blautia sp. DC 3652 | Leuconostoc mesenteroides subsp. | 61.7% |
| | | cremoris NRIC 1838 | |
| 53 | Blautia sp. DC 3652 | Lactobacillus parabuchneri | 61.5% |
| | | NBRC 107865 | |
| 54 | Blautia sp. DC 3652 | Lactobacillus porcinae JCM 19617 | 61.4% |
| 55 | Blautia sp. DC 3652 | Oenococcus ceni DSM 20282 | 61.3% |
| 56 | Blautia sp. DC 3652 | Lactobacillus sp. NRIC 1029 | 60.8% |
| 57 | Blautia sp. DC 3652 | Lactobacillus saniviri JCM 17471 | 60.8% |
| 58 | Blautia sp. DC 3652 | Lactobacillus alimentarius | 60.4% |
| | | NBRC 106464 | |
| 59 | Blautia sp. DC 3652 | Lactobacillus paralimentarius | 60.1% |
| | | NBRC 107152 | |
| 60 | Blautia sp. DC 3652 | Sporolactobacillus inulinus | 60.0% |
| | | NRIC 1133 | |
| 61 | Blautia sp. DC 3652 | Lactobacillus salivarius subsp. | 59.7% |
| | | salicinius NRIC 1072 | |
| 62 | Blautia sp. DC 3652 | Lactobacillus ozensis JCM 17196 | 59.1% |
| 63 | Blautia sp. DC 3652 | Lactobacillus sakei subsp. carnosus | 59.1% |
| | | NBRC 107868 | |
| 64 | Blautia sp. DC 3652 | Lactobacillus bifermentans | 59.0% |
| | | JCM 1094 | |
| 65 | Blautia sp. DC 3652 | Lactobacillus hammesii JCM 16170 | 58.8% |
| 66 | Blautia sp. DC 3652 | Lactobacillus suebicus JCM 9504 | 58.8% |
| 67 | Blautia sp. DC 3652 | Lactobacillus brantas DSM 23927 | 58.5% |
| 68 | Blautia sp. DC 3652 | Lactobacillus dioliverans | 58.4% |
| | | NBRC 107869 | |
| 69 | Blautia sp. DC 3652 | Lactobacillus satsumensis | 58.2% |
| | | JCM 12392 | |
| 70 | Blautia sp. DC 3652 | Weissella halotolerans NRIC 1627 | 57.3% |
| 71 | Blautia sp. DC 3652 | Lactobacillus sharpeae JCM 1186 | 57.2% |
| 72 | Blautia sp. DC 3652 | Lactobacillus otakiensis JCM 15040 | 57.0% |
| 73 | Blautia sp. DC 3652 | Lactobacillus nasuensis JCM 17158 | 56.7% |
| 74 | Blautia sp. DC 3652 | Lactobacillus parafarraginis | 56.6% |
| | | JCM 14109 | |
| 75 | Blautia sp. DC 3652 | Lactobacillus pobuzihii | 56.5% |
| | | NBRC 103219 | |
| 76 | Blautia sp. DC 3652 | Lactobacillus ruminis NBRC 102161 | 56.4% |
| 77 | Blautia sp. DC 3652 | Lactobacillus colechominis | 56.1% |
| | | JCM 11550 | |
| 78 | Blautia sp. DC 3652 | Lactobacillus delbrueckii | 56.1% |
| | | NBRC 102622 | |
| 79 | Blautia sp. DC 3652 | Enterococcus hirae NRIC 102 | 55.6% |
| 80 | Blautia sp. DC 3652 | Osnococcus ceni ATCC 27311 | 55.6% |

TABLE 1-3

| No. | * 1 | * 2 | * 3 |
|---|---|---|---|
| 81 | Blautia sp. DC 3652 | Lactobacillus iwatensis JCM 18838 | 55.5% |
| 82 | Blautia sp. DC 3652 | Lactobacillus farraginis JCM 14108 | 55.5% |
| 83 | Blautia sp. DC 3652 | Lactobacillus malefermentans | 55.5% |
| | | NRIC 1081 | |
| 84 | Blautia sp. DC 3652 | Pediococcus claussenii JCM 18046 | 54.7% |
| 85 | Blautia sp. DC 3652 | Lactobacillus nagelii JCM 12492 | 54.4% |
| 86 | Blautia sp. DC 3652 | Lactobacillus equigenerosi | 53.5% |
| | | JCM 14505 | |
| 87 | Blautia sp. DC 3652 | Lactobacillus fructivorans NRIC 224 | 53.4% |
| 88 | Blautia sp. DC 3652 | Lactobacillus agilis JCM 1187 | 53.2% |
| 89 | Blautia sp. DC 3652 | Lactobacillus acetotoferans | 53.2% |
| | | JCM 3825 | |
| 90 | Blautia sp. DC 3652 | Lactobacillus kisonensis JCM 15041 | 52.6% |
| 91 | Blautia sp. DC 3652 | Lactobacillus malefermentans | 52.1% |
| | | DSM 5705 | |
| 92 | Blautia sp. DC 3652 | Lactobacillus florum JCM 16035 | 51.6% |
| 93 | Blautia sp. DC 3652 | Enterococcus avium NITE BP-03386 | 51.6% |
| 94 | Blautia sp. DC 3652 | Lactobacillus apodemi JCM 16172 | 51.5% |
| 95 | Blautia sp. DC 3652 | Lactobacillus plantarum IFO 3070 | 51.2% |
| 96 | Blautia sp. DC 3652 | Lactobacillus paracollinoides | 50.8% |
| | | JCM 11969 | |
| 97 | Blautia sp. DC 3652 | Lactobacillus gastricus JCM 15952 | 50.7% |
| 98 | Blautia sp. DC 3652 | Lactobacillus fujiensis JCM 16395 | 50.1% |
| 99 | Blautia sp. DC 3652 | Lactobacillus ghanensis JCM 15611 | 50.1% |
| 100 | Blautia sp. DC 3652 | Lactobacillus pentosiphilus | 50.0% |
| | | JCM 31145 | |
| 101 | Blautia sp. DC 3652 | Lactobacillus faecalis subsp. | 49.9% |
| | | liquefaciens NRIC 1748 | |
| 102 | Blautia sp. DC 3652 | Lactobacillus buchneri NRIC 1082 | 49.9% |
| 103 | Blautia sp. DC 3652 | Lactobacillus hilgardii NBRC 15865 | 49.7% |
| 104 | Blautia sp. DC 3652 | Lactobacillus paralimentarius | 49.7% |
| | | NBRC 106486 | |
| 105 | Blautia sp. DC 3652 | Lactobacillus gasseri JCM 1131 | 49.6% |
| 106 | Blautia sp. DC 3652 | Lactobacillus plantarum NRIC 1068 | 49.6% |
| 107 | Blautia sp. DC 3652 | Lactobacillus acidophilus IFO 13951 | 49.6% |
| 108 | Blautia sp. DC 3652 | Lactobacillus pentosus IFO 12011 | 49.2% |
| 109 | Blautia sp. DC 3652 | Lactobacillus amylolyticus | 49.1% |
| | | JCM 12529 | |
| 110 | Blautia sp. DC 3652 | Lactobacillus senmaizukei | 48.3% |
| | | NBRC 103853 | |
| 111 | Blautia sp. DC 3652 | Lactobacillus equi JCM 10991 | 48.3% |
| 112 | Blautia sp. DC 3652 | Pediococcus inopinatus JCM 12518 | 48.3% |
| 113 | Blautia sp. DC 3652 | Lactobacillus hamsteri JCM 6256 | 48.2% |
| 114 | Blautia sp. DC 3652 | Lactobacillus sucicole JCM 15457 | 48.0% |
| 115 | Blautia sp. DC 3652 | Fructobacillus fructosus NBRC 3516 | 47.9% |
| 116 | Blautia sp. DC 3652 | Lactobacillus camelliae JCM 13995 | 47.9% |
| 117 | Blautia sp. DC 3652 | Lactobacillus curvatus NBRC 15884 | 47.7% |
| 118 | Blautia sp. DC 3652 | Lactobacillus hardei JCM 16179 | 47.5% |
| 119 | Blautia sp. DC 3652 | Lactobacillus hayakitensis | 47.2% |
| | | JCM 14209 | |
| 120 | Blautia sp. DC 3652 | Lactobacillus iners JCM 12513 | 47.0% |

TABLE 1-4

| No. | * 1 | * 2 | * 3 |
|---|---|---|---|
| 121 | Blautia sp. DC 3652 | Lactobacillus aviarius subsp. aviarius | 47.0% |
| | | NBRC 102162 | |
| 122 | Blautia sp. DC 3652 | Lactobacillus plantarum subsp. | 46.9% |
| | | plantarum NBRC 15891 | |
| 123 | Blautia sp. DC 3652 | Lactobacillus floricola JCM 16512 | 46.8% |
| 124 | Blautia sp. DC 3652 | Lactobacillus equicursoris JCM 14600 | 46.5% |
| 125 | Blautia sp. DC 3652 | Lactobacillus collinoides NRIC 1049 | 46.3% |
| 126 | Blautia sp. DC 3652 | Lactobacillus vini JCM 14280 | 46.2% |
| 127 | Blautia sp. DC 3652 | Lactobacillus versmoldensis | 46.0% |
| | | NBRC 106069 | |
| 128 | Blautia sp. DC 3652 | Lactobacillus panis JCM 11053 | 45.8% |
| 129 | Blautia sp. DC 3652 | Lactobacillus koresnsis JCM 16448 | 45.7% |
| 130 | Blautia sp. DC 3652 | Lactobacillus frumenti JCM 11122 | 45.3% |
| 131 | Blautia sp. DC 3652 | Lactobacillus delbrueckii subsp. | 45.3% |
| | | delbrueckii IFO 3538 | |
| 132 | Blautia sp. DC 3652 | Lactobacillus parakefiri NBRC 15890 | 44.9% |
| 133 | Blautia sp. DC 3652 | Lactobacillus fuchuensis JCM 11249 | 44.4% |
| 134 | Blautia sp. DC 3652 | Lactobacillus delbrueckii subsp. lactis | 44.4% |
| | | NRIC 1081 | |
| 135 | Blautia sp. DC 3652 | Tetragenococcus halophilus subsp. | 44.3% |
| | | halophilus NBRC 100498 | |
| 136 | Blautia sp. DC 3652 | Enterococcus caccae DSM 19114 | 44.3% |
| 137 | Blautia sp. DC 3652 | Lactobacillus nenjiangensis | 44.3% |
| | | JCM 30919 | |
| 138 | Blautia sp. DC 3652 | Streptococcus alactolyticus | 44.2% |
| | | DSM 100950 | |
| 139 | Blautia sp. DC 3652 | Pediococcus cellicola JCM 14152 | 44.1% |
| 140 | Blautia sp. DC 3652 | Leucoriostoc dextranicum AHU 1078 | 43.3% |
| 141 | Blautia sp. DC 3652 | Lactobacillus garvieae NBRC 100934 | 43.1% |
| 142 | Blautia sp. DC 3652 | Lactobacillus uvarum JCM 16870 | 42.9% |
| 143 | Blautia sp. DC 3652 | Lactobacillus thailandensis | 42.5% |
| | | JCM 13996 | |
| 144 | Blautia sp. DC 3652 | Lactobacillus kefiranofaciens subsp. | 42.5% |
| | | kefirgranum JCM 8572 | |
| 145 | Blautia sp. DC 3652 | Lactobacillus spicheri NBRC 107185 | 42.3% |
| 146 | Blautia sp. DC 3652 | Lactobacillus algidus JCM 10491 | 42.2% |
| 147 | Blautia sp. DC 3652 | Lactobacillus kimchiensis JCM 17702 | 42.2% |
| 148 | Blautia sp. DC 3652 | Lactobacillus aquaticus JCM 16869 | 42.2% |
| 149 | Blautia sp. DC 3652 | Lactobacillus johnsonii JCM 2012 | 41.7% |
| 150 | Blautia sp. DC 3652 | Lactobacillus kitasatonis JCM 1039 | 40.8% |
| 151 | Blautia sp. DC 3652 | Lactobacillus rapi NBRC 109618 | 39.5% |
| 152 | Blautia sp. DC 3652 | Lactobacillus manihotivorans | 39.3% |
| | | JCM 12514 | |
| 153 | Blautia sp. DC 3652 | Lactobacillus ceti JCM 15609 | 39.2% |
| 154 | Blautia sp. DC 3652 | Lactobacillus delbrueckii susp. | 39.1% |
| | | indicus JCM 15610 | |
| 155 | Blautia sp. DC 3652 | Lactobacillus taiwanensis | 38.7% |
| | | NBRC 109049 | |
| 156 | Blautia sp. DC 3652 | Lactobacillus mali NRIC 1078 | 38.6% |
| 157 | Blautia sp. DC 3652 | Lactobacillus ultunensis JCM 16177 | 38.1% |
| 158 | Blautia sp. DC 3652 | Lactobacillus hilgardii NRIC 1060 | 37.6% |
| 159 | Blautia sp. DC 3652 | Lactobacillus rhamnosus IFO 3425 | 37.2% |

TABLE 1-5

| No. | *1 | *2 | *3 |
|---|---|---|---|
| 160 | Blautia sp. DC 3652 | Lactobacillus amylotrophicus | 37.1% |
| | | JCM 1124 | |
| 161 | Blautia sp. DC 3652 | Weissella confusa NBRC 106489 | 36.9% |
| 162 | Blautia sp. DC 3652 | Lactobacillus plantarum DSM 13273 | 33.8% |
| 163 | Blautia sp. DC 3652 | Pediococcus parvulus JCM 5889 | 33.4% |
| 164 | Blautia sp. DC 3652 | Lactobacillus ingluviei JCM 12531 | 33.3% |
| 165 | Blautia sp. DC 3652 | Lactobacillus delbrueckii subsp. | 33.2% |
| | | lactis JCM 1557 | |
| 166 | Blautia sp. DC 3652 | Lactococcus lactis subsp. lactis | 32.9% |
| | | NRIC 1149 | |
| 167 | Blautia sp. DC 3652 | Lactobacillus nantensis NBRC 107153 | 32.9% |
| 168 | Blautia sp. DC 3652 | Lactobacillus paucivorans JCM 18045 | 32.4% |
| 169 | Blautia sp. DC 3652 | Lactobacillus sanfranciscesnsis | 31.9% |
| | | JCM 5668 | |
| 170 | Blautia sp. DC 3652 | Lactobacillus pontis JCM 11051 | 31.8% |
| 171 | Blautia sp. DC 3652 | Lactobacillus namurensis | 30.7% |
| | | NBRC 107158 | |
| 172 | Blautia sp. DC 3652 | Lactobacillus intestinalis JCM 7548 | 30.5% |
| 173 | Blautia sp. DC 3652 | Enterococcus hirae JCM 8717 | 30.2% |
| 174 | Blautia sp. DC 3652 | Enterococcus hirae JCM 8719 | 30.2% |
| 175 | Blautia sp. DC 3652 | Lactococcus lactis subsp. tructae | 30.0% |
| | | DSM 21502 | |
| 176 | Blautia sp. DC 3652 | Lactobacillus jensenii JCM 15953 | 29.7% |
| 177 | Blautia sp. DC 3652 | Lactococcus lactis subsp. lactis | 29.6% |
| | | NRIC 1074 | |
| 178 | Blautia sp. DC 3652 | Lactobacillus porci DSM 105804 | 29.2% |
| 179 | Blautia sp. DC 3652 | Enterococcus hirae NRIC 108 | 29.1% |
| 180 | Blautia sp. DC 3652 | Lactobacillus mindensis | 29.0% |
| | | NBRC 107162 | |
| 181 | Blautia sp. DC 3652 | Lactobacillus hokkaidonensis | 28.8% |
| | | JCM 18461 | |
| 182 | Blautia sp. DC 3652 | Lactobacillus hominis DSM 23910 | 28.4% |
| 183 | Blautia sp. DC 3652 | Enterococcus avium NITE BP-03387 | 28.4% |
| 184 | Blautia sp. DC 3652 | Pediococcus damnosus JCM 5886 | 28.3% |
| 185 | Blautia sp. DC 3652 | Lactobacillus kalixensis JCM 15954 | 28.2% |
| 186 | Blautia sp. DC 3652 | Lactobacillus buchneri NRIC 1079 | 28.0% |
| 187 | Blautia sp. DC 3652 | Pediococcus pentosaceus IFO 3891 | 27.8% |
| 188 | Blautia sp. DC 3652 | Lactobacillus oligofermentans | 27.7% |
| | | JCM 16175 | |
| 189 | Blautia sp. DC 3652 | Pediococcus acidilactici NRIC 1102 | 27.5% |
| 190 | Blautia sp. DC 3652 | Lactobacillus murinus IFO 14221 | 27.4% |
| 191 | Blautia sp. DC 3652 | Lactobacillus amylophilus IFO 15881 | 26.6% |
| 192 | Blautia sp. DC 3652 | Lactobacillus wasatchensis | 26.4% |
| | | DSM 29958 | |
| 193 | Blautia sp. DC 3652 | Lactobacillus delbrueckii subsp. | 26.2% |
| | | delbrueckii IAM 1149 | |
| 194 | Blautia sp. DC 3652 | Lactobacillus secaliphilus JCM 15613 | 25.8% |
| 195 | Blautia sp. DC 3652 | Pediccoccus pentosaceus NRIC 1106 | 25.0% |
| 196 | Blautia sp. DC 3652 | Lactobacillus delbrueckii subsp. | 24.6% |
| | | lactis IFO 3073 | |
| 197 | Blautia sp. DC 3652 | Clostridium bolteae NITE BP-03384 | 63.0% |
| 198 | Blautia sp. DC 3652 | Escherichia fergusoni NITE BP-03388 | 60.2% |
| 199 | Blautia sp. DC 3652 | Escherichia fergusoni NITE BP-03389 | 58.2% |
| 200 | Blautia sp. DC 3652 | Anaerofustis stercorihominis | 57.2% |
| | | DSM 17244 | |

TABLE 1-6

| No. | * 1 | * 2 | * 3 |
|---|---|---|---|
| 201 | Blautia sp. DC 3652 | Hungatella sp. NITE BP-03385 | 53.9% |
| 202 | Blautia sp. DC 3652 | Clostridium clostridioforme JCM 1291 | 52.8% |
| 203 | Blautia sp. DC 3652 | Escherichia fergusonii NITE BP-03390 | 52.1% |
| 204 | Blautia sp. DC 3652 | Anaerotruncus colihominis DSM 17241 | 50.0% |
| 205 | Blautia sp. DC 3652 | Solobacterium moorei DSM 22971 | 48.2% |
| 206 | Blautia sp. DC 3652 | Eubacterium ramulus DSM 16296 | 45.9% |
| 207 | Blautia sp. DC 3652 | Escherichia coli ATCC 27325 | 45.8% |
| 208 | Blautia sp. DC 3652 | Faecalicoccus pleomorphus | 45.8% |
| | | NITE BP-03393 | |
| 209 | Blautia sp. DC 3652 | Hungatella hathewayi NITE BP-03395 | 43.9% |
| 210 | Blautia sp. DC 3652 | Hungatella effluvii DSM 24995 | 43.2% |
| 211 | Blautia sp. DC 3652 | Eubacterium limosum JCM 6501 | 41.6% |
| 212 | Blautia sp. DC 3652 | Bacteroides faecichinchillae | 41.3% |
| | | DSM 26883 | |
| 213 | Blautia sp. DC 3652 | Intestinimonas butyriciproducens | 40.2% |
| | | NITE BP-03397 | |
| 214 | Blautia sp. DC 3652 | Clostridium hathewayi DSM 13479 | 40.1% |
| 215 | Blautia sp. DC 3652 | Finegoldia magna JCM 1766 | 39.4% |
| 216 | Blautia sp. DC 3652 | Dielma fastidiosa DSM 26099 | 38.8% |
| 217 | Blautia sp. DC 3652 | Hungatella hathewayi NITE BP-03396 | 38.6% |
| 218 | Blautia sp. DC 3652 | Intestinimonas butyriciproducens | 38.5% |
| | | NITE BP-03399 | |
| 219 | Blautia sp. DC 3652 | Faecalicoccus pleomorphus | 38.3% |
| | | NITE BP-03392 | |
| 220 | Blautia sp. DC 3652 | Clostridium bolteae NITE BP-03383 | 37.9% |
| 221 | Blautia sp. DC 3652 | Akkermansia muciniphila DSM 26127 | 35.6% |
| 222 | Blautia sp. DC 3652 | Arcobacter butzleri DSM 107942 | 35.3% |
| 223 | Blautia sp. DC 3652 | Prevotella rara DSM 105141 | 34.0% |
| 224 | Blautia sp. DC 3652 | Sutterella megalosphaeroides | 32.3% |
| | | DSM 106861 | |
| 225 | Blautia sp. DC 3652 | Hungatella sp. NITE BP-03398 | 31.6% |
| 226 | Blautia sp. DC 3652 | Faecalicoccus pleomorphus | 30.5% |
| | | NITE BP-03391 | |
| 227 | Blautia sp. DC 3652 | Akkermansia muciniphila DSM 22959 | 29.8% |
| 228 | Blautia sp. DC 3652 | Coprobacillus cateniformis | 29.5% |
| | | DSM 15921 | |
| 229 | Blautia sp. DC 3652 | Bacteroides stercoris DSM 19555 | 29.5% |
| 230 | Blautia sp. DC 3652 | Faecalicoccus sp. NITE BP-03394 | 28.0% |
| 231 | Blautia sp. DC 3652 | Parascardovia denticolens JCM 12538 | 26.8% |
| 232 | Blautia sp. DC 3652 | Clostridium hathewayi DSM 13480 | 26.2% |
| 233 | Blautia sp. DC 3652 | Clostridium celerscresens JCM 15734 | 25.4% |
| 234 | Blautia sp. DC 3652 | Bifidobacterium coryneforme | 69.1% |
| | | JCM 5819 | |
| 235 | Blautia sp. DC 3652 | Bifidobacterium psychraeraphilum | 62.7% |
| | | JCM 15958 | |
| 236 | Blautia sp. DC 3652 | Bifidobacterium pullorum JCM 1214 | 51.0% |
| 237 | Blautia sp. DC 3652 | Bifidobacterium bifidum JCM 1255 | 48.0% |
| 238 | Blautia sp. DC 3652 | Bifidobacterium indicum JCM 1302 | 45.5% |
| 239 | Blautia sp. DC 3652 | Bifidobacterium adolescentis | 36.3% |
| | | JCM 1275 | |
| 240 | Blautia sp. DC 3652 | Bifidobacterium actinocoloniiforme | 36.1% |
| | | JCM 18048 | |

TABLE 1-7

| No. | * 1 | * 2 | * 3 |
|---|---|---|---|
| 241 | Blautia sp. DC 3652 | Bifidobacterium thermacidophilum | 34.8% |
| | | subsp. thermacidophilum | |
| | | JCM 11165 | |
| 242 | Blautia sp. DC 3652 | Bifidobacterium ruminantium | 34.8% |
| | | JCM 8222 | |
| 243 | Blautia sp. DC 3652 | Bifidobacterium callitrichos | 33.6% |
| | | JCM 17296 | |
| 244 | Blautia sp. DC 3652 | Bifidobacterium longum subsp. | 32.6% |
| | | longum JCM 1217 | |
| 245 | Blautia sp. DC 3652 | Bifidobacterium scardovii JCM 12489 | 32.1% |
| 246 | Blautia sp. DC 3652 | Bifidobacterium longum subsp. | 30.9% |
| | | suis JCM 1269 | |
| 247 | Blautia sp. DC 3652 | Bifidobacterium gallinarum JCM 6291 | 27.8% |
| 248 | Blautia sp. DC 3652 | Bifidobacterium reuteri JCM 17295 | 27.7% |
| 249 | Blautia sp. DC 3652 | Bifidobacterium saeculare JCM 8223 | 26.2% |
| 250 | Blautia sp. DC 3652 | Bifidobacterium magnum JCM 1218 | 25.7% |
| 251 | Blautia sp. DC 3652 | Bifidobacterium animalis subsp. | 24.8% |
| | | animalis JCM 1190 | |
| 252 | Blautia sp. DC 3652 | Bifidobacterium stellenboschense | 24.7% |
| | | JCM 17298 | |

TABLE 1-8

| No. | * 1 | * 2 | * 3 |
|---|---|---|---|
| 253 | Blautia sp. DC 3652 | Klebsiella aerogenes DSM 30053 | 94.0% |
| 254 | Blautia sp. DC 3652 | Clostridium sporogenes JCM 1416 | 91.4% |
| 255 | Blautia sp. DC 3652 | Edwardsiella tarda NBRC 105688 | 89.6% |
| 256 | Blautia sp. DC 3652 | Lactobacillus plajomi NBRC 107333 | 89.2% |
| 257 | Blautia sp. DC 3652 | Megasphaera elsdenii JCM 1772 | 80.5% |
| 258 | Blautia sp. DC 3652 | Lactobacillus chiayiensis NBRC 112906 | 78.4% |
| 259 | Blautia sp. DC 3652 | Lactobacillus ixorae NBRC 111239 | 76.6% |
| 260 | Blautia sp. DC 3652 | Yersinia bercovieri NBRC 105717 | 73.5% |
| 261 | Blautia sp. DC 3652 | Lactobacillus modestisalitolerans | 72.2% |
| | | NBRC 107235 | |
| 262 | Blautia sp. DC 3652 | Clostridioides difficile JCM 1298 | 66.5% |
| 263 | Blautia sp. DC 3652 | Yersinia rohdei NBRC 105715 | 60.5% |
| 264 | Blautia sp. DC 3652 | Lactobacillus kullabergensis DSM 26262 | 58.0% |
| 265 | Blautia sp. DC 3652 | Ruminococcus gnavus JCM 6515 | 56.8% |
| 266 | Blautia sp. DC 3652 | Providencia alcalifaciens NBRC 105687 | 53.9% |
| 267 | Blautia sp. DC 3652 | Bifidobacterium catenulatum subsp. | 49.4% |
| | | catenulatum DSM 16992 | |
| 268 | Blautia sp. DC 3652 | Clostridium ramosum JCM 1298 | 48.3% |
| 269 | Blautia sp. DC 3652 | Lactobacillus mellifer DSM 26254 | 41.1% |
| 270 | Blautia sp. DC 3652 | Anaerostipes caccae JCM 13470 | 39.4% |
| 271 | Blautia sp. DC 3652 | Bifidobacterium primatium DSM 100687 | 38.8% |
| 272 | Blautia sp. DO 3652 | Bifidobacterium simiarum DSM 103153 | 37.7% |
| 273 | Blautia sp. DC 3652 | Chitinophaga skermanii NBRC 109753 | 37.5% |
| 274 | Blautia sp. DC 3652 | Parabacteroides distasonis JCM 5825 | 34.9% |
| 275 | Blautia sp. DC 3652 | Bifidobacterium catulorum DSM 103164 | 32.2% |
| 276 | Blautia sp. DC 3652 | Lactobacillus apinorum DSM 28257 | 30.4% |
| 277 | Blautia sp. DC 3652 | Prevotella melaninogenica JCM 6325 | 28.2% |
| 278 | Blautia sp. DC 3652 | Clostridium paraputrificum JCM 1293 | 27.9% |
| 279 | Blautia sp. DC 3652 | Bifidobacterium jacchi DSM 103362 | 26.6% |
| 280 | Blautia sp. DC 3652 | Lactobacillus suantsaiihabitans | 26.4% |
| | | NBRC 113532 | |
| 281 | Blautia sp. DC 3652 | Cryptobacterium sp. NITE BP-03476 | 26.3% |
| 282 | Blautia sp. DC 3652 | Lacrimispora sphenoides JCM 1415 | 26.2% |
| 283 | Blautia sp. DC 3652 | Citrobacter sedlakii NBRC 105722 | 25.4% |

Method for Producing 8-Prenylnaringenin from Isoxanthohumol (2)

### Comparative Example 2

After isoxanthohumol (final concentration of 50 mg/L) was added to Wilkins-Chalgren Anaerobe Broth (available from Thermo Fisher Scientific), the mixture was heated and sterilized, the gas phase was replaced with a gas of N₂:CO₂:H₂ (80%: 10%: 10%). and the resulting product was used as a fermentation medium. Eubacterium limosum JCM 6421 strain was inoculated as the first microorganism to the fermentation medium and subjected to anaerobic culture at 37° C. for 72 hours. After the culture, 5 mL of the culture solution was diluted in 3 times its volume of ethanol and filtered through a 0.45 μm filter, and the filtrate was then subjected to quantitative analysis of 8-prenylnaringenin by HPLC under the following conditions. No second microorganism was used.

HPLC Conditions
Column: Inertsil ODS-3 (4.6 mm×25 cm, 5 μm)
Eluent A: H₂O/Formic acid (99/1)
Eluent B: Acetonitrile/Formic acid (99/1)
Gradient condition: Solution B: 0 to 5 min (20%), then 10 min (70%), and then 30 min (70%)
Column temperature: 40° C.
Flow rate: 1.0 mL/min
Detection: UV at 290 nm

### Example 2

The analysis was performed in the same manner as in Comparative Example 2 except that in addition to the first microorganism, microorganisms described in Table 2 were inoculated as a second microorganism into the fermentation medium.

Conversion rates of isoxanthohumol to 8-prenylnaringenin are shown in Table 2. No. 1 in the table corresponds to Comparative Example 2.

In the tables, “*1” represents the first microorganism, “*2” represents the second microorganism, and “*3” represents the conversion rates from isoxanthohumol to 8-prenylnaringenin.

TABLE 2

| No. | * 1 | * 2 | * 3 |
|---|---|---|---|
| 1 | Eubacterium limosum | — | 0.1% |
| | JCM 6421 | | |
| 2 | Eubacterium limosum | Escherichia coli | 41.7% |
| | JCM 6421 | ATCC 27325 | |
| 3 | Eubacterium limosum | Bifidobacterium bifidum | 10.7% |
| | JCM 6421 | JCM1255 | |
| 4 | Eubacterium limosum | Anaerofustis stercorihominis | 10.1% |
| | JCM 6421 | DSM 17244 | |
| 5 | Eubacterium limosum | Bifidobacterium pullorum | 7.0% |
| | JCM 6421 | JCM1214 | |
| 6 | Eubacterium limosum | Bifidobacterium coryneforme | 1.3% |
| | JCM 6421 | JCM 5819 | |

Method for Producing 6-Hydroxydaidzein from Glycitein

### Comparative Example 3

After glycitein (final concentration of 50 mg/L) was added to Wilkins-Chalgren Anaerobe broth (available from Thermo Fisher Scientific), the mixture was heated and sterilized, the gas phase was replaced with a gas of N₂:CO₂:H₂ (80%:10%:10%), and the resulting product was used as a fermentation medium. Blautia sp. DC 3652 (NITE BP-02924) strain or Eubacterium limosum JCM 6421 strain was inoculated as the first microorganism to the fermentation medium and subjected to anaerobic culture at 37° C. for 72 hours. After the culture, 5 mL of the culture solution was diluted in 3 times its volume of ethanol and filtered through a 0.45 μm filter, and the filtrate was then subjected to quantitative analysis of 6-hydroxydaidzein by HPLC under the following conditions. No second microorganism was used.

HPLC Conditions
Column: Inertsil ODS-3 (4.6 mm×25 cm, 5 μm)
Eluent A: H₂O/Formic acid (99/1)
Eluent B: Acetonitrile/Formic acid (99/1)
Gradient condition: Solution B; 0 to 5 min (20%).then 10 min (50%), and then 25 min (50%)
Column temperature: 40° C.
Flow rate: 1.0 mL/min
Detection: UV at 280 nm

### Example 3

The analysis was performed in the same manner as in Comparative Example 3 except that in addition to the first microorganism, microorganisms described in Table 3 were inoculated as a second microorganism into the fermentation medium.

Conversion rates of glycitein to 6-hydroxydaidzein are shown in Table 3. No. 1 and No. 7 in the table correspond to Comparative Example 3.

Note that in the table, “*I” represents the first microorganism. “*2” represents the second microorganism, and “*3” represents the conversion rate of glycitein to 6-hydroxydaidzein.

TABLE 3

| No. | * 1 | * 2 | * 3 |
|---|---|---|---|
| 1 | Blautia sp. DC 3652 | — | 16.4% |
| 2 | Blautia sp. DC 3652 | Weissella confusa DSM 20196 | 86.8% |
| 3 | Blautia sp. DC 3652 | Streptococcus uberis NRIC 1153 | 86.8% |
| 4 | Blautia sp. DC 3652 | Escherichia coli ATCC 27325 | 40.7% |
| 5 | Blautia sp. DC 3652 | Bifidobacterium psychraerophilum | 28.0% |
| | | JCM 15958 | |
| 6 | Blautia sp. DC 3652 | Bifidobacterium coryneforme | 23.0% |
| | | JCM 5819 | |
| 7 | Eubacterium limosum | — | 3.3% |
| | JCM 6421 | | |
| 8 | Eubacterium limosum | Bifidobacterium coryneforme | 10.1% |
| | JCM 6421 | JCM 5819 | |
| 9 | Eubacterium limosum | Escherichia coli ATCC 27325 | 5.3% |
| | JCM 6421 | | |
| 10 | Eubacterium limosum | Weissella confusa DSM 20188 | 4.8% |
| | JCM 6421 | | |

Method for Producing Eriodictyol from Hesperetin

### Comparative Example 4

After hesperetin (final concentration of 250 mg/L or 100 mg/L) was added to Wilkins-Chalgren Anaerobe broth (available from Thermo Fisher Scientific), the mixture was heated and sterilized, the gas phase was replaced with a gas of N₂:CO₂:H₂ (80%:10%:10%), and the resulting product was used as a fermentation medium. Blautia sp. DC 3652 (NITE BP-02924) strain or Eubacterium limosum JCM 6421 strain was inoculated as the first microorganism to the fermentation medium and subjected to anaerobic culture at 37° C. for 72 hours. After the culture, 5 mL of the culture solution was diluted in 3 times its volume of ethanol and filtered through a 0.45 μm filter, and the filtrate was then subjected to quantitative analysis of eriodictyol by HPLC under the following conditions.

HPLC Conditions
Column: Inertsil ODS-3 (4.6 mm×25 cm, 5 μm)
Eluent A: H₂O/Formic acid (99/1)
Eluent B: Acetonitrile/Formic acid (99/1)
Gradient condition; Solution B: 0 to 5 min (20%), then 10 min (50%), and then 30 min (50%)
Column temperature: 40° C.
Flow rate: 1.0 mL/min
Detection: UV at 280 nm

### Example 4

The analysis was performed in the same manner as in Comparative Example 4 except that in addition to the first microorganism, microorganisms described in Table 4 were inoculated as a second microorganism into the fermentation medium.

Conversion rates from hesperetin to eriodictyol are shown in Table 4. No. 1 and No. 8 in the table correspond to Comparative Example 4.

Note that in the table. “*1” represents the first microorganism, “*2” represents the second microorganism, “*3” represents the final concentration (mg/L) of hesperetin, and “*4” represents the conversion rate of hesperetin to eriodictyol.

TABLE 4

| No. | * 1 | * 2 | * 3 | * 4 |
|---|---|---|---|---|
| 1 | Blautia sp. DC 3652 | — | 250 | 39.9% |
| 2 | Blautia sp. DC 3652 | Anaerofustis stercorihominis | 250 | 52.7% |
| | | DSM 17244 | | |
| 3 | Blautia sp. DC 3652 | Weissella confusa | 250 | 49.9% |
| | | DSM 20196 | | |
| 4 | Blautia sp. DC 3652 | Lactobacillus brevis | 250 | 48.3% |
| | | NRIC 1037 | | |
| 5 | Blautia sp. DC 3652 | Leuconostoc dextranicum | 250 | 44.8% |
| | | IFO3347 | | |
| 6 | Blautia sp. DC 3652 | Bifidobacterium coryneforme | 250 | 43.9% |
| | | JCM 5819 | | |
| 7 | Blautia sp. DC 3652 | Streptococcus uberis | 250 | 43.1% |
| | | NRIC 1153 | | |
| 8 | Eubacterium limosum | — | 100 | 10.1% |
| | JCM 6421 | | | |
| 9 | Eubacterium limosum | Weissella confusa | 100 | 46.3% |
| | JCM 6421 | DSM 20186 | | |
| 10 | Eubacterium limosum | Anaerofustis stercorihominis | 100 | 26.9% |
| | JCM 6421 | DSM 17244 | | |
| 11 | Eubacterium limosum | Bifidobacterium coryneforme | 100 | 23.9% |
| | JCM 6421 | JCM 5819 | | |
| 12 | Eubacterium limosum | Bifidobacterium | 100 | 19.6% |
| | JCM 6421 | psychraerophilum | | |
| | | JCM 15988 | | |
| 13 | Eubacterium limosum | Escherichia coli | 100 | 18.3% |
| | JCM 6421 | ATCC 27325 | | |
| 14 | Eubacterium limosum | Streptococcus uberis | 100 | 15.3% |
| | JCM 6421 | NRIC 1153 | | |

Method for Producing Esculetin from Scoparone

### Comparative Example 5

After scoparone (final concentration of 50 mg/L or 100 mg/L) was added to Wilkins-Chalgren Anaerobe broth (available from Thermo Fisher Scientific), the mixture was heated and sterilized, the gas phase was replaced with a gas of N₂:CO₂:H₂ (80%:10%:10%), and the resulting product was used as a fermentation medium. Blautia sp. DC 3652 (NITE BP-02924) strain or Eubacterium limosum JCM 6421 strain was inoculated as the first microorganism to the fermentation medium and subjected to anaerobic culture at 37° C. for 72 hours. After the culture, 5 mL of the culture solution was diluted in 3 times its volume of ethanol and filtered through a 0.45 μm filter, and the filtrate was then subjected to quantitative analysis of esculetin by HPLC under the following conditions.

HPLC Conditions
Column: Inertsil ODS-3 (4.6 mm×25 cm, 5 μm)
Eluent A: H₂O/Formic acid (99/1)
Eluent B: Acetonitrile/Formic acid (99/1)
Gradient condition: Solution B; 0 to 5 min (20%), then 10 min (70%), and then 30 min (70%)
Column temperature: 40° C.
Flow rate: 1.0 mL/min
Detection: UV at 340 nm

### Example 5

The analysis was performed in the same manner as in Comparative Example 5 except that in addition to the first microorganism, microorganisms described in Table 5 were inoculated as a second microorganism into the fermentation medium.

Conversion rates of scoparone to esculetin are shown in Table 5. No. 1 and No. 10 in the table correspond to Comparative Example 5.

Note that in the table. “*1” represents the first microorganism, “*2” represents the second microorganism, “*3” represents the final concentration (mg/L) of scoparone, and “*4” represents the conversion rate of scoparone to esculetin.

TABLE 5

| No. | * 1 | * 2 | * 3 | * 4 |
|---|---|---|---|---|
| 1 | Blautia sp. DC 3652 | — | 50 | 6.1% |
| 2 | Blautia sp. DC 3652 | Escherichia coli ATCC 27325 | 50 | 89.8% |
| 3 | Blautia sp. DC 3652 | Lactobacillus brevis | 50 | 61.7% |
| | | NRIC 1037 | | |
| 4 | Blautia sp. DC 3652 | Leuconostoc dextranicum | 50 | 24.1% |
| | | IFO3347 | | |
| 5 | Blautia sp. DC 3652 | Weissella confusa DSM 20195 | 50 | 17.9% |
| 6 | Blautia sp. DC 3652 | Streptococcus uberis | | 13.4% |
| | | NRIC 1153 | | |
| 7 | Blautia sp. DC 3652 | Bifidobacterium coryneforme | 50 | 7.9% |
| | | JCM 5819 | | |
| 8 | Blautia sp. DC 3652 | Bifidobacterium | 50 | 7.5% |
| | | psychraerophilum | | |
| | | JCM 18958 | | |
| 9 | Blautia sp. DC 3652 | Anaerofustis stercorihominis | 50 | 7.2% |
| | | DSM 17244 | | |
| 10 | Eubacterium limosum | — | 100 | 14.4% |
| | JCM 6421 | | | |
| 11 | Eubacterium limosum | Lactobacillus brevis | 100 | 92.5% |
| | JCM 6421 | NRIC 1037 | | |
| 12 | Eubacterium limosum | Clostridium bolteae | 100 | 91.8% |
| | JCM 6421 | NITE BP-03384 | | |
| 13 | Eubacterium limosum | Weissella confusa DSM 20196 | 100 | 57.3% |
| | JCM 6421 | | | |
| 14 | Eubacterium limosum | Escherichia coli ATCC 27326 | 100 | 38.8% |
| | JCM 6421 | | | |
| 15 | Eubacterium limosum | Anaerofustis stercorihominis | 100 | 38.7% |
| | JCM 6421 | DSM 17244 | | |
| 16 | Eubacterium limosum | Bifidobacterium coryneforme | 100 | 35.9% |
| | JCM 6421 | JCM 5819 | | |

Method for Producing 4-Acetyl Resorcinol from Paeonol

### Comparative Example 6

After paeonol (final concentration of 50 mg/L) was added to a modified GAM medium (available from Nissui Pharma Medical Sales Co Ltd), the mixture was heated and sterilized, the gas phase was replaced with a gas of N₂:CO₂:H₂ (80%:10%:10%), and the resulting product was used as a fermentation medium. Blautia sp. DC 3652 (NITE BP-02924) strain was inoculated as a first microorganism to the fermentation medium and subjected to anaerobic culture at 37° C. for 72 hours. After the culture, 5 mL of the culture solution was diluted in 3 times its volume of ethanol and filtered through a 0.45 μm filter, and the filtrate was then subjected to quantitative analysis of 4-acetylresorcinol by HPLC under the following conditions.

HPLC Conditions
Column: Inertsil ODS-3 (4.6 mm×25 cm, 5 μm)
Eluent A: H₂O/Formic acid (99/1)
Eluent B: Acetonitrile/Formic acid (99/1)
Gradient condition: Solution B; 0 to 5 min (20%), then 10 min (70%), and then 30 min (70%)
Column temperature: 40° C.
Flow rate: 1.0 mL/min
Detection: UV at 280 nm

### Example 6

The analysis was performed in the same manner as in Comparative Example 6 except that in addition to the first microorganism, microorganisms described in Table 6 were inoculated as a second microorganism into the fermentation medium.

Conversion rates of paeonol to 4-acetylresorcinol are shown in Table 6. No. 1 in the table corresponds to Comparative Example 6.

Note that in the table, “*1” represents the first microorganism, “*2” represents the second microorganism, and “*3” represents the conversion rate of paeonol to 4-acetylresorcinol.

TABLE 6

| No. | * 1 | * 2 | * 3 |
|---|---|---|---|
| 1 | Blautia sp. DC 3652 | — | 2.2% |
| 2 | Blautia sp. DC 3652 | Escherichia coli ATCC 27325 | 4.8% |

Confirmation of Effect of Promoting Demethylation of 8-Prenylnaringenin from Isoxanthohumol by Co-culture with E. coli Monogene-Deficient Strain

### Example 7

After isoxanthohumol (final concentration of 50 mg/L) was added to a modified GAM medium (available from Nissui Pharma Medical Sales Co Ltd), the mixture was heated and sterilized, the gas phase was replaced with a gas of N₂:CO₂:H₂ (80%:10%:10%), and the resulting product was used as a fermentation medium. To the fermentation medium, Blautia sp. DC 3652 (NITE BP-02924) strain was inoculated as a first microorganism, and Escherichia coli BW25113 strain or a monogene-deficient strain thereof (KO Collection) was inoculated as a second microorganism, followed by anaerobic culture at 37° C. for 3 days. After the culture, 5 mL of the culture solution was diluted in 3 times its volume of ethanol and filtered through a 0.45 μm filter, and the filtrate was then subjected to quantitative analysis of 8-prenylnaringenin by HPLC under the following conditions.

HPLC Conditions
Column: Inertsil ODS-3 (4.6 mm×25 cm, 5 μm)
Eluent A: H₂O/Formic acid (99/1)
Eluent B: Acetonitrile/Formic acid (99/1)
Gradient condition: Solution B; 0 to 5 min (20%), then 10 min (70%), and then 30 min (70%)
Column temperature: 40° C.
Flow rate: 1.0 mL/min
Detection: UV at 290 nm

Conversion rates of isoxanthohumol to 8-prenylnaringenin are shown in Table 7.

Note that in the table, “*1” represents the first microorganism. “*2” represents the second microorganism, “*3” represents the deficient gene, “*4” represents the conversion rate from isoxanthohumol to 8-prenvinaringenin, “2d” represents results obtained after 2 days of culture, and “3d” represents results obtained after 3 days of culture.

TABLE 7

| | | | | *4 | |
|---|---|---|---|---|---|
| No. | * 1 | * 2 | * 3 | 2 d | 3 d |
| 1 | Blautia sp. DC3652 | — | — | 16.9% | 24.8% |
| 2 | Blautia sp. DC3652 | Escherichia coli | — | 75.1% | 84.8% |
| | | BW25113 | | | |
| 3 | Blautia sp. DC3652 | Escherichia coli | thy A | 19.9% | 24.4% |
| | | JW2795 | | | |
| 4 | Blautia sp. DC3652 | Escherichia coli | gcy T | | 73.8% |
| | | JW2873 | | | |
| 5 | Blautia sp. DC3652 | Escherichia coli | gly A | 54.5% | 74.5% |
| | | JW2535 | | | |
| 6 | Blautia sp. DC3652 | Escherichia coli | ygf A | | 77.4% |
| | | JW2879 | | | |
| 7 | Blautia sp. DC3652 | Escherichia coli | met F | | 69.9% |
| | | JW3913 | | | |
| 8 | Blautia sp. DC3652 | Escherichia coli | met H | | 86.8% |
| | | JW3979 | | | |
| 9 | Blautia sp. DC3652 | Escherichia coli | pur T | | 72.0% |
| | | JW1838 | | | |
| 10 | Blautia sp. DC3652 | Escherichia coli | pur N | 78.0% | |
| | | JW2485 | | | |
| 11 | Blautia sp. DC3652 | Escherichia coli | pur U | 73.4% | |
| | | JW1220 | | | |

At least the thyA (dihydrofolate reductase)-deficient strain (No. 3) and the glyA (glycine hydroxymethyltransferase)-deficient strain (No. 5) had no effect of promoting demethylation as compared with the non-deficient strain (No. 2), and thus it was presumed that at least these genes promoted regeneration of tetrahydrofolic acid (THF).

1. A method for producing a demethylated compound, comprising co-culturing, in a solution containing a compound with one or more methoxy groups in one or more side chains, a microorganism having a demethylation ability of eliminating one or more methyl groups of one or more methoxy groups from a compound with the one or more methoxy groups in one or more side chains, and a microorganism having an activity to promote the demethylation, to produce the demethylated compound in which one or more methyl groups of one or more methoxy groups have eliminated from the compound with the one or more methoxy groups in the one or more side chain.

2. The method according to claim 1, wherein the microorganism having the activity to promote the demethylation is one or more microorganisms selected from the group consisting of a microorganism belonging to lactic acid bacterium, a microorganism belonging to the genus Akkermansia, a microorganism belonging to the genus Anaerofustis, a microorganism belonging to the genus Anaerotruncus, a microorganism belonging to the genus Arcobacter, a microorganism belonging to the genus Bacteroides, a microorganism belonging to the genus Clostridium, a microorganism belonging to the genus Coprobacillus, a microorganism belonging to the genus Dielma, a microorganism belonging to the genus Escherichia, a microorganism belonging to the genus Eubacterium, a microorganism belonging to the genus Faecalicoccus, a microorganism belonging to the genus Finegoldia, a microorganism belonging to the genus Hungatella, a microorganism belonging to the genus Intestinimonas, a microorganism belonging to the genus Parascardovia, a microorganism belonging to the genus Prevotella, a microorganism belonging to the genus Solobacterium, a microorganism belonging to the genus Sutterella, a microorganism belonging to the genus Bifidobacterium, a microorganism belonging to the genus Anaerostipes, a microorganism belonging to the genus Chitinophaga, a microorganism belonging to the genus Citrobacter, a microorganism belonging to the genus Clostridioides, a microorganism belonging to the genus Cryptobacterium, a microorganism belonging to the genus Edwardsiella, a microorganism belonging to the genus Klebsiella, a microorganism belonging to the genus Lacrimispora, a microorganism belonging to the genus Megasphaera, a microorganism belonging to the genus Parabacteroides, a microorganism belonging to the genus Providencia, a microorganism belonging to the genus Ruminococcus, and a microorganism belonging to the genus Yersinia.

3. The method according to claim 2, wherein the microorganism belonging to lactic acid bacterium is one or more microorganisms selected from the group consisting of a microorganism belonging to the genus Carnobacterium, a microorganism belonging to the genus Enterococcus, a microorganism belonging to the genus Fructobacillus, a microorganism belonging to the genus Lactobacillus, a microorganism belonging to the genus Lactococcus, a microorganism belonging to the genus Leuconostoc, a microorganism belonging to the genus Oenococcus, a microorganism belonging to the genus Pediococcus, a microorganism belonging to the genus Sporolactobacillus, a microorganism belonging to the genus Streptococcus, a microorganism belonging to the genus Tetragenococcus, and a microorganism belonging to the genus Weissella.

4. The method according to claim 1, wherein the compound with the one or more methoxy groups in the one or more side chain is a polyphenol with one or more methoxy groups in one or more side chains, and the demethylated compound in which the one or more methyl groups of the one or more methoxy groups has eliminated is a demethylated polyphenol in which one or more methyl groups of the one or more methoxy groups have eliminated.

5. The method according to claim 4, wherein a combination of the polyphenol with the one or more methoxy groups in the one or more side chains and the demethylated polyphenol in which the one or more methyl groups of the one or more methoxy groups have eliminated is one or more combinations selected from the group consisting of
a combination of flavanone with one or more methoxy groups in one or more side chains and demethylated flavanone in which one or more methyl groups of the one or more methoxy groups have eliminated,
a combination of isoflavone with one or more methoxy groups in one or more side chains and demethylated isoflavone in which one or more methyl groups of the one or more methoxy groups have eliminated,
a combination of coumarin with one or more methoxy groups in one or more side chains and demethylated coumarin in which one or more methyl groups of the one or more methoxy groups have eliminated, and
a combination of simple phenols with one or more methoxy groups in one or more side chains and demethylated simple phenols in which one or more methyl groups of the one or more methoxy groups have eliminated.

6. The method according to claim 5, wherein the combination of the flavanone with the one or more methoxy groups in the one or more side chain and the demethylated flavanone in which the one or more methyl groups of the one or more methoxy groups have eliminated is one or more combinations selected from the group consisting of a combination of isoxanthohumol and 8-prenylnaringenin, and a combination of hesperetin and eriodictyol.

7. The method according to claim 5, wherein the isoflavone with the one or more methoxy groups in the one or more side chains is glycitein, and the demethylated isoflavone in which the one or more methyl groups of the one or more methoxy groups have eliminated is 6-hydroxydaidzein.

8. The method according to claim 5, wherein the coumarin with the one or more methoxy groups in the one or more side chains is scoparone, and the demethylated coumarin in which the one or more methyl groups of the one or more methoxy groups have eliminated is one or more compounds selected from the group consisting of esculetin, scopoletin, and isoscopoletin.

9. The method according to claim 5, wherein the simple phenols with the one or more methoxy groups in the one or more side chain is paeonol, and the demethylated simple phenols in which the one or more methyl groups of the one or more methoxy groups have eliminated is 4-acetylresorcinol.

10. The method according to claim 1, wherein the microorganism having the activity to promote the demethylation is a microorganism having an activity to promote regeneration of tetrahydrofolic acid from 5-methyltetrahydrofolate.

11. The method according to claim 10, wherein the microorganism having the activity to promote regeneration of tetrahydrofolic acid from 5-methyltetrahydrofolate is a microorganism producing dihydrofolate reductase-thymidylate synthase (E.C.1.5.1.3) and/or glycine hydroxymethyltransferase (E.C.2.1.2.1).

12. A method for promoting production of a demethylated compound, comprising co-culturing, in a solution containing a compound with one or more methoxy groups in one or more side chains, a microorganism having a demethylation ability of eliminating one or more methyl groups of one or more methoxy groups from a compound with the one or more methoxy groups in one or more side chains, and a microorganism having an activity to promote the demethylation, to promote production of the demethylated compound in which one or more methyl groups of one or more methoxy groups have eliminated from the compound with the one or more methoxy groups in the one or more side chains.
